(19) United States
(12) Patent Application Publication
(10) Pub. No.: US 2020/0357507 A1
(43) Pub. Date: Nov. 12, 2020

(54) GENERATING AND PRESENTING GRAPH DATA STRUCTURES REPRESENTING PATIENT VISIT PATHS AND PHYSICIAN REFERRAL NETWORKS

(71) Applicant: KOMODO HEALTH, San Francisco, CA (US)

(72) Inventors: Benjamin James Campbell Blalock, Astoria, NY (US); Alexander Graham Glenday, Brooklyn, NY (US); Jason Richard Prestinario, Brooklyn, NY (US)

(73) Assignee: KOMODO HEALTH, San Francisco, CA (US)

(21) Appl. No.: 16/406,754

(22) Filed: May 8, 2019

(51) Int. Cl. G16H 40/20; G16H 10/60; G06F 16/22

(52) U.S. Cl. CPC G16H 40/20; G06F 16/2237; G16H 10/60

(57) ABSTRACT

Techniques for generating and presenting graph data structures representing patient visit paths and physician referral networks are disclosed. A system generates one or more graph data structures representing one or more referred visit paths and/or inferred visit paths. Based on the graph data structures representing the visit paths, the system generates another graph data structure representing a physician network. A user interface presents a graph representing the physician network. Based on the presented graph, a user may thereby determine influential levels of different physicians for referring one or more patients to a particular physician.

### TECHNICAL FIELD

The present disclosure relates to graph data structures. In particular, the present disclosure relates to generating and presenting graph data structures representing patient visit paths and physician referral networks.

### BACKGROUND

Patients are referred to physicians in various ways. One way to refer a patient to a physician is through an explicit referral. An explicit referral is an official referral that a physician (a “referring physician”) gives to a patient to see another physician (a “referee physician”). As an example, a primary care physician and/or a general practitioner may refer a patient to a specialist physician. As another example, a physician of one specialty may refer a patient to a physician of another specialty. An explicit referral may be recorded in a patient's medical records. Additionally or alternatively, an explicit referral may be submitted to an insurance company to support the patient's insurance claim for the visit to the referee physician. The insurance company may deny insurance coverage for the visit to the referee physician without the explicit referral. Different organizations (e.g., health care providers and insurance companies) may use different databases, systems, and/or data formats for recording and/or storing information on explicit referrals.

However, other less definitive ways of referring a patient to a physician may be used. Such referrals may be referred to as “implicit referrals.” As an example, during a patient visit, a physician may mention the name and/or department of another physician to the patient. As another example, a physician may provide a list of other physicians to a patient, and the patient may choose any of the physicians on the list. Implicit referrals are generally not tracked in any database or system.

Existing network analysis systems are not able to track explicit referrals and implicit referrals accurately. Hence, it is difficult to accurately determine, for example, a chain of referrals that lead a patient to a particular physician, and/or physicians that have particular influential power in referring patients to a particular physician.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a graph generation system, in accordance with one or more embodiments;

FIG. 2 illustrates an example set of operations for generating and presenting patient visit paths and physician referral networks based on a set of insurance claims, in accordance with one or more embodiments;

FIGS. 3A-B illustrate an example set of operations for generating a graph data structure representing a patient visit path based on explicit referrals, in accordance with one or more embodiments;

FIGS. 4A-B illustrate an example set of operations for generating a graph data structure representing a patient visit path based on implicit referrals, in accordance with one or more embodiments;

FIG. 5 illustrates an example set of operations for generating a graph data structure representing a physician referral network, in accordance with one or more embodiments;

FIGS. 6A-I illustrate an example for generating patient visit paths and physician referral network, in accordance with one or more embodiments;

FIG. 7A illustrates an example of a graph, presented on a user interface, representing a physician referral network, in accordance with one or more embodiments;

FIG. 7B illustrates an example of a graph, presented on a user interface, representing a relational network on medical departments, in accordance with one or more embodiments; and

FIG. 8 shows a block diagram that illustrates a computer system in accordance with one or more embodiments.

### DETAILED DESCRIPTION

In the following description, for the purposes of explanation, numerous specific details are set forth in order to provide a thorough understanding. One or more embodiments may be practiced without these specific details. Features described in one embodiment may be combined with features described in a different embodiment. In some examples, well-known structures and devices are described with reference to a block diagram form in order to avoid unnecessarily obscuring the present invention.
1. GENERAL OVERVIEW
2. GRAPH GENERATION SYSTEM ARCHITECTURE
3. GENERATING AND PRESENTING GRAPH DATA STRUCTURES REPRESENTING PATIENT VISIT PATHS AND PHYSICIAN REFERRAL NETWORKS
4. EXAMPLE EMBODIMENT
5. HARDWARE OVERVIEW
6. MISCELLANEOUS; EXTENSIONS

### 1. GENERAL OVERVIEW

One or more embodiments involve the use of a graph data structure. A graph data structure stores a set of objects in which some pairs of objects are connected or related. An object in a graph data structure may be referred to as a “vertex.” A connection between two vertices may be referred to as an “edge.” Where the order of a pair of vertices matters (that is, A→B is different than B→A), then a connection between the two vertices may be referred to as a “directed edge.” A graph data structure may be stored and/or presented as a set of tables. Additionally or alternatively, a graph data structure may be stored and/or presented in diagrammatic form.

One or more embodiments include generating a graph data structure representing a patient visit path based on explicit referrals. A patient visit path based on explicit referrals may also be referred to as a “referred visit path.” A set of data structures representing a plurality of patient visit records (also referred to as “visit records”) are analyzed. Pairwise comparison is performed on the visit records to identify 2-tuples of visit records matching a particular set of criteria. The criteria may include (a) the visit records must reference a same patient; (b) a visit time of the visit record in the first element of the 2-tuple must be before or simultaneous with a visit time of the visit record in the second element of the 2-tuple; and (c) an attending physician of the visit record in the first element of the 2-tuple must be same as a referring physician of the visit record in the second element of the 2-tuple. Responsive to determining that a 2-tuple of visit records matches the criteria, a graph generation system connects, in a graph data structure, (a) a first vertex representing the visit record in the first element of the 2-tuple and (b) a second vertex representing the visit record in the second element of the 2-tuple. The connection forms a directed edge from the first vertex to the second vertex. Visit records represented by connected vertices may be referred to as being connected on a patient visit path.

One or more embodiments include generating a graph data structure representing a patient visit path based on implicit referrals. A patient visit path based on implicit referrals may also be referred to as an “inferred visit path.” A set of data structures representing a plurality of patient visit records (also referred to as “visit records”) are analyzed. Pairwise comparison is performed on the visit records to identify 2-tuples of visit records matching a particular set of criteria. The criteria may include (a) the visit records must reference a same patient; (b) a visit time of the visit record in the first element of the 2-tuple must be before a visit time of the visit record in the second element of the 2-tuple; and (c) a difference between the two visit times must be below a threshold value. Responsive to determining that a 2-tuple of visit records matches the criteria, a graph generation system connects, in a graph data structure, (a) a first vertex representing the visit record in the first element of the 2-tuple and (b) a second vertex representing the visit record in the second element of the 2-tuple. The connection forms a directed edge from the first vertex to the second vertex. Visit records represented by connected vertices may be referred to as being connected on a patient visit path.

One or more embodiments include generating a graph data structure representing a physician referral network (also referred to as a “physician network”). The graph data structure representing the physician referral network is generated based on a graph data structure representing one or more patient visit paths. A graph data structure representing a patient visit path is analyzed to determine that a first visit record is connected to a second visit record on a patient visit path. Responsive to determining the physicians indicated by the first visit record and the second visit record, a graph generation system connects, in a data structure representing a physician network, (a) a first vertex representing a first physician indicated by the first visit record and (b) a second vertex representing a second physician indicated by the second visit record. The connection forms a directed edge from the first vertex to the second vertex. Physicians represented by connected vertices may be referred to as being connected in a physician referral network Similar operations may be performed to generate a graph data structure representing a provider referral network (also referred to as a “provider network”). One or more embodiments include presenting, on a user interface, a graph representing a physician referral network. A graph data structure representing a physician referral network is analyzed. The graph data structure includes vertices representing physicians. The graph data structure includes edges representing an explicit and/or an implicit referral from one physician to another physician. A user interface presents a set of nodes representing the physicians. The user interface presents links between the nodes representing the explicit and/or implicit referrals between the physicians Similar operations may be performed to present, on a user interface, a graph representing a provider referral network.

In one or more embodiments, a network analysis system generates and analyzes graph data structures representing visit paths and/or physician networks. The network analysis system takes into account information associated with explicit referrals and/or implicit referrals to determine one or more referral networks. The network analysis presents a graph representing a referral network on a user interface. Hence, for example, a user may determine a chain of visits preceding a visit to a particular physician. As another example, a user may determine a chain of referrals that lead a patient to a particular physician. As another example, a user may determine physicians that have particular influential power in referring patients to a particular physician.

One or more embodiments described in this Specification and/or recited in the claims may not be included in this General Overview section.

### 2. GRAPH GENERATION SYSTEM ARCHITECTURE

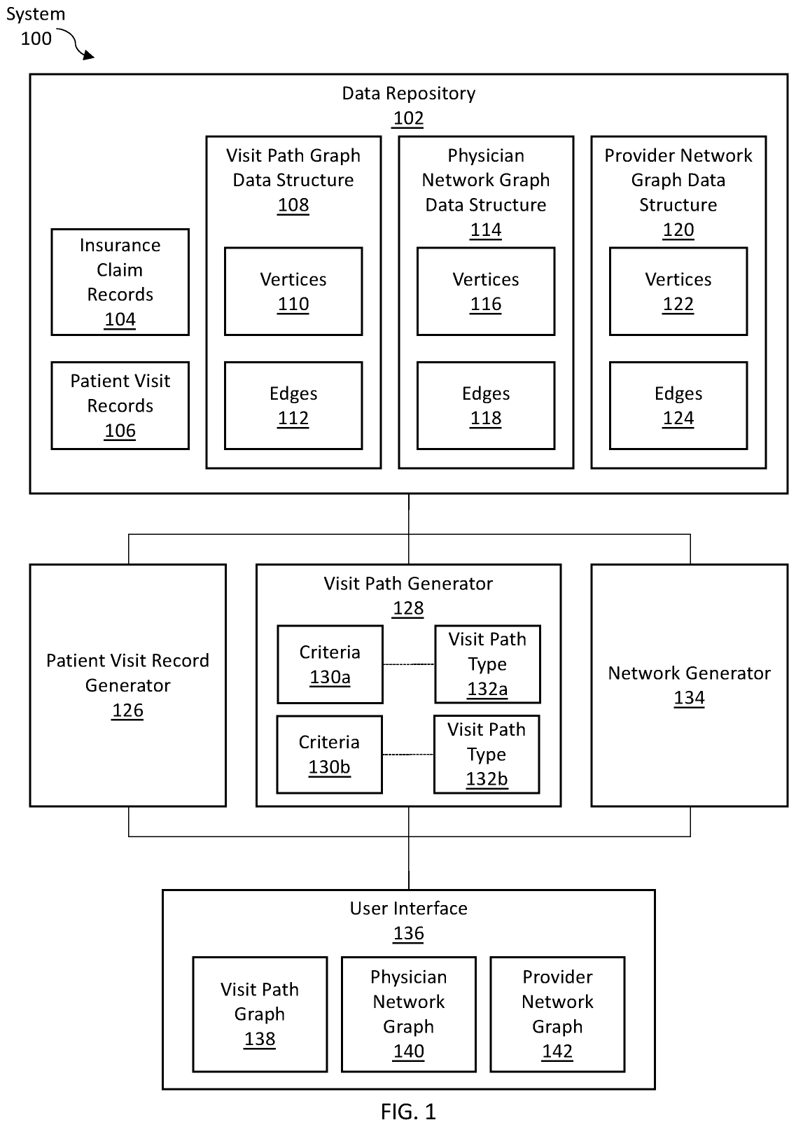

FIG. 1 illustrates a graph generation system, in accordance with one or more embodiments. As illustrated in FIG. 1, a system 100 includes a data repository 102, a patient visit record generator 126, a visit path generator 128, a network generator 134, and a user interface 136. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as a patient visit record generator 126, visit path generator 128, and/or network generator 134. Alternatively or additionally, a data repository 102 may be implemented or executed on a computing system separate from a patient visit record generator 126, visit path generator 128, and/or network generator 134. The data repository 102 may be communicatively coupled to the patient visit record generator 126, visit path generator 128, and/or network generator 134 via a direct connection or via a network.

Information describing insurance claim records 104, patient visit records 106, one or more visit path graph data structures 108, one or more physician network graph data structures 114, and/or one or more provider network graph data structures 120 may be implemented across any of components within the system 100. However, this information is illustrated within the data repository 102 for purposes of clarity and explanation.

In one or more embodiments, an insurance claim record 104 is a record of a claim, for submission to an insurance company, requesting insurance coverage for a patient visit with a physician. Insurance claim records 104 may be obtained from a variety of data sources. Additionally or alternatively, insurance claim records 104 obtained from various data sources may be stored in various data structures.

A system 100 may pre-process insurance claim records 104 obtained from various sources to convert the insurance claim records 104 into a standardized format and/or schema. Examples of schemas used for insurance claims are the 837P (Professional) data format and the Form CMS-1500 data format. A schema for insurance claim records 104 may include fields such as a patient identifier (ID), patient name, patient birthday, visit date, claim date, diagnosis, procedure, attending physician ID, referring physician ID, healthcare provider ID, and/or healthcare provider location. A schema for insurance claim records 104 may include using codes for certain fields. As an example, a diagnosis field may accept only a limited set of diagnosis codes. Diagnosis codes map to different medical diagnoses. As another example, a procedure field may accept only a limited set of procedure codes. Procedure codes map to different medical procedures.

Insurance claim records 104 are stored in a data repository 102 in any of a variety of data structures. One data structure suitable for storing insurance claim records is a columnar data storage format, such as Apache Hadoop's Parquet, RCFile, and ORC. According to a columnar data storage format, values in each column may be physically stored in contiguous memory locations. According to a columnar data storage format, compression may be performed column by column. Compression techniques specific to a data type may be applied as column values tend to be of the same data type.

Insurance claim records 104 obtained from different sources may reference a same object and/or thing in different ways. As an example, one source may address physicians using one system of IDs. Another source may address physicians using another system of IDs. Additionally or alternatively, insurance claim records 104 obtained from different sources may include different subsets of information about patient visits. As an example, one source may include a patient's name and social security number (SSN), without including driver license number. Another source may include a patient's name and driver license number, without including SSN.

In one or more embodiments, a patient visit record 106 (also referred to as a “visit record”) is a record, corresponding to a patient visit, compiled from one or more insurance claim records 104. One or more insurance claim records 104 may correspond to the same patient visit. As an example, multiple insurance claim records may correspond to the same patient visit when multiple insurance claims are filed for the same visit. As another example, multiple insurance claim records may correspond to the same patient visit when different insurance claims are filed with different insurance companies for the same visit. Various techniques may be used to identify a group of insurance claim records 104 corresponding to the same patient visit. An example of such technique is entity resolution. A visit record 106 includes aggregated information from one or more insurance claim records 104 corresponding to the same patient visit.

As an example, one insurance claim record may indicate that a patient ID is 123, an attending physician ID is 456, and a visit date is Mar. 1, 2019. Another insurance claim record may indicate that a patient ID is 123, an attending physician ID is 456, and a diagnosis code is 789. The information from the two claim records may be aggregated into a single visit record. The visit record may indicate that a particular patient visit is associated with a patient ID 123, an attending physician ID 456, a visit date Mar. 1, 2019, and a diagnosis code 789.

A visit record 106 may be stored using a same standardized format and/or schema (such as the 837P (Professional) data format) as an insurance claim record 104. Alternatively, a visit record 106 may be stored using a different format and/or schema than an insurance claim record 104. A visit record 106 may be stored using a same type of data structure (such as a columnar data storage format) as an insurance claim record 104. Alternatively, a visit record 106 may be stored using a different type of data structure than an insurance claim record 104.

In one or more embodiments, a graph data structure (such as any of visit path graph data structure 108, physician network graph data structure 110, and/or provider network graph data structure 112) stores a set of objects in which some pairs of objects are connected or related. An object in a graph data structure may be referred to as a “vertex.” A connection between two vertices may be referred to as an “edge.” Each edge is a “directed edge,” such that the order of a pair of vertices matters (that is, A→B is different than B→A). A graph data structure may be stored and/or presented as a set of tables. Additionally or alternatively, a graph data structure may be stored and/or presented in diagrammatic form.

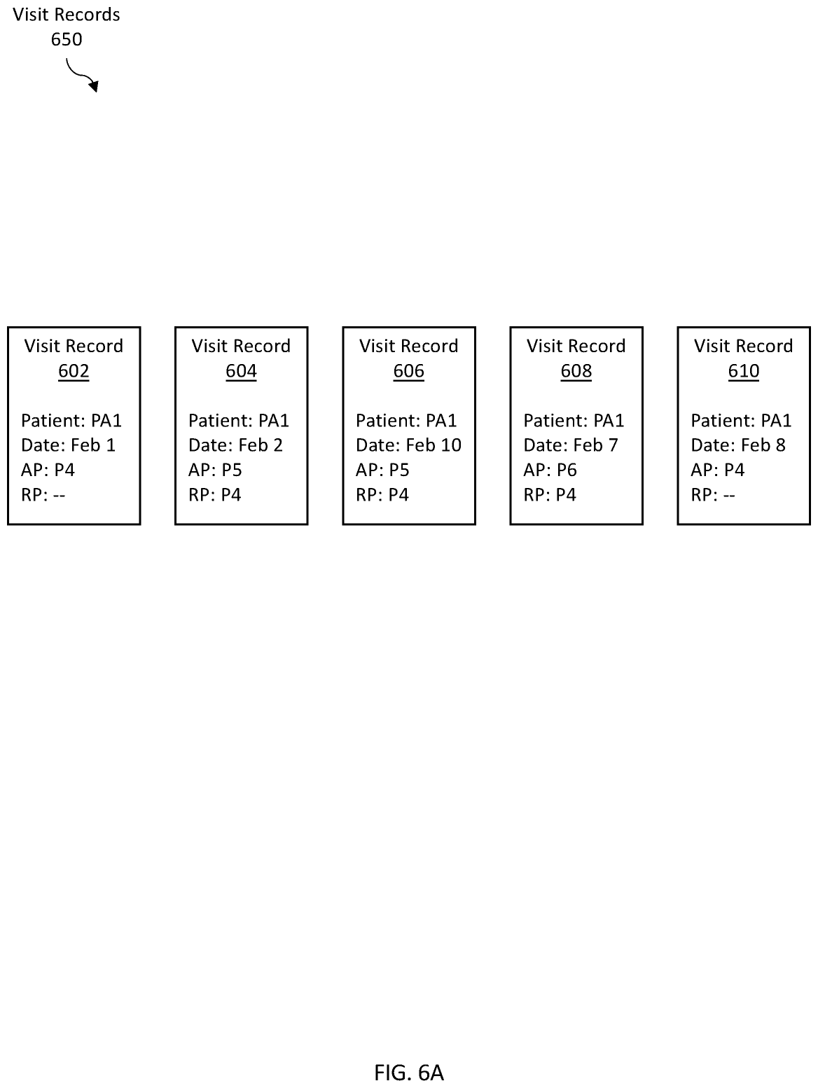

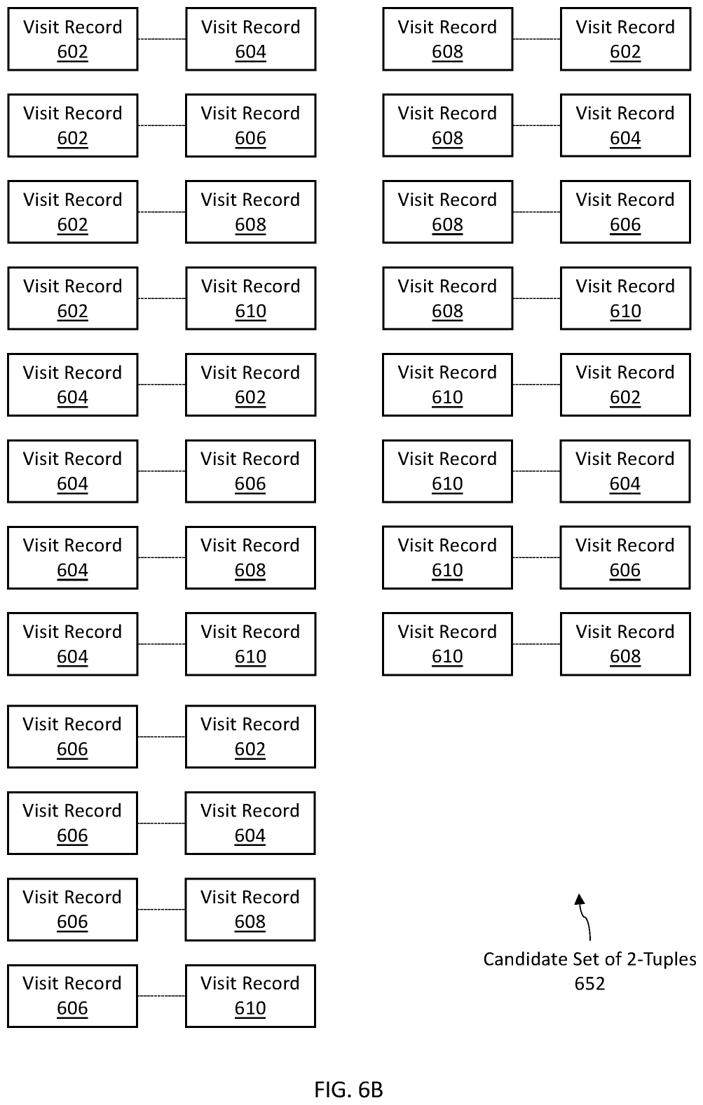

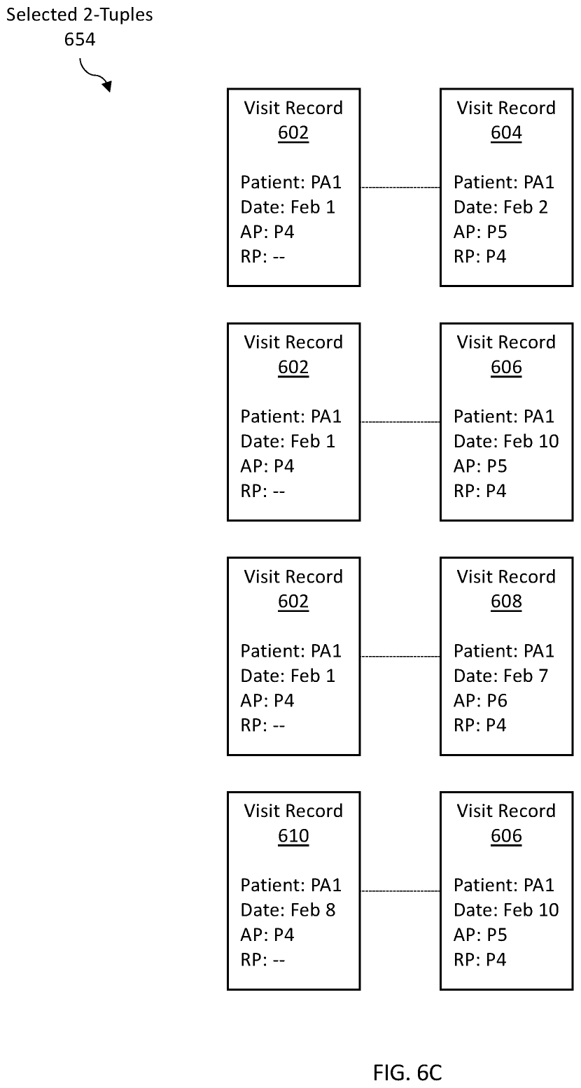

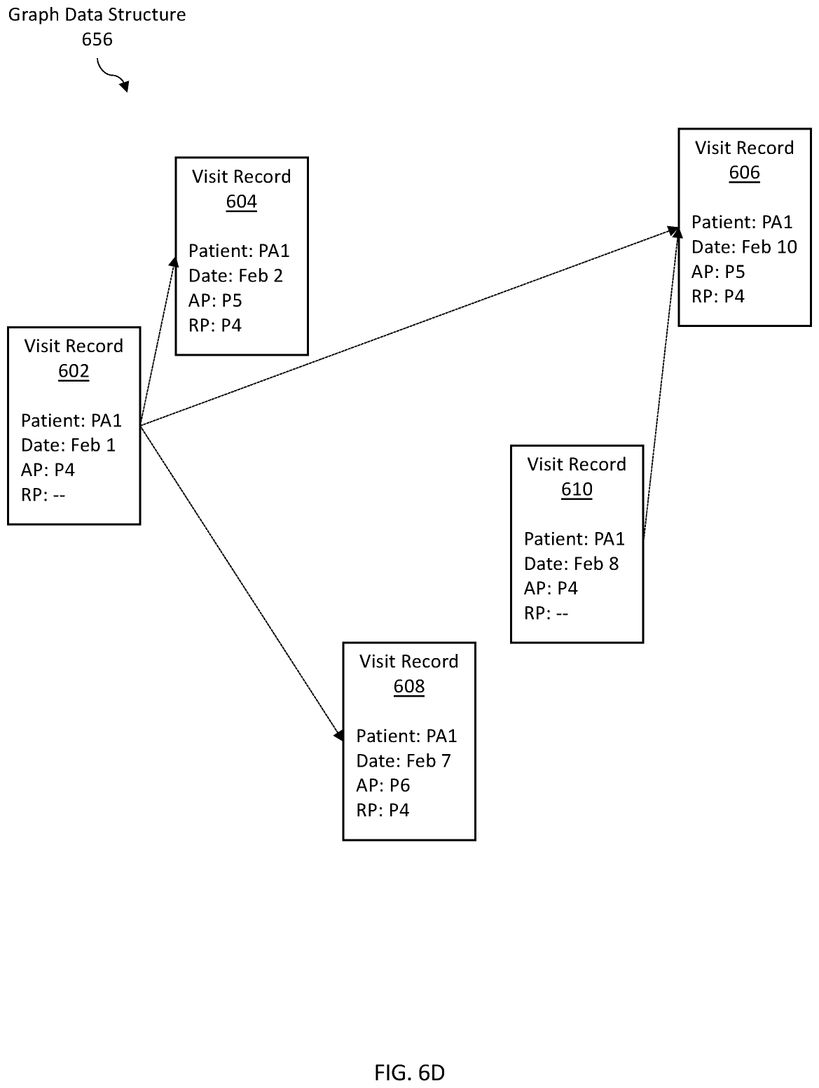

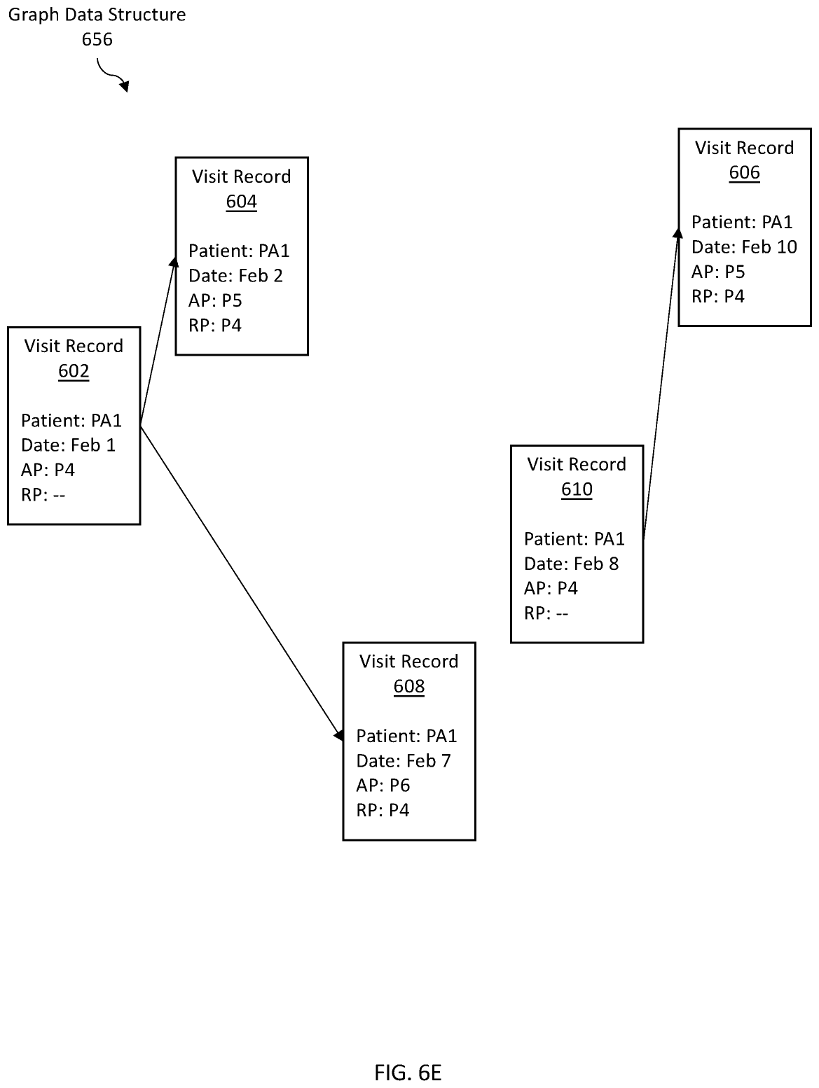

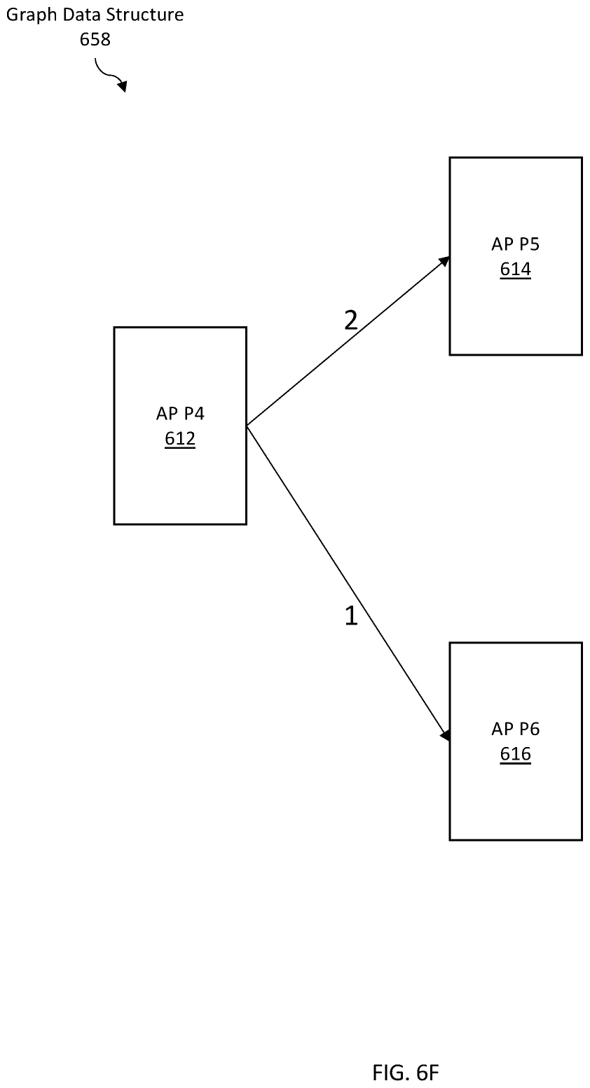

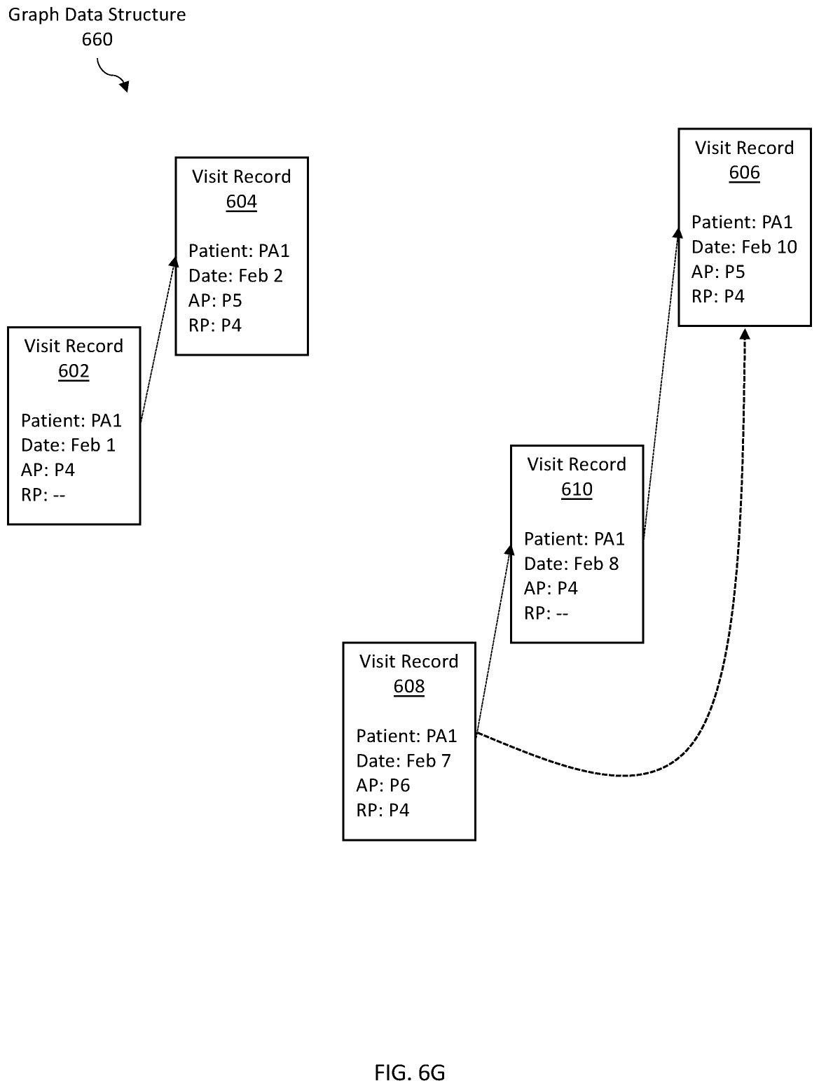

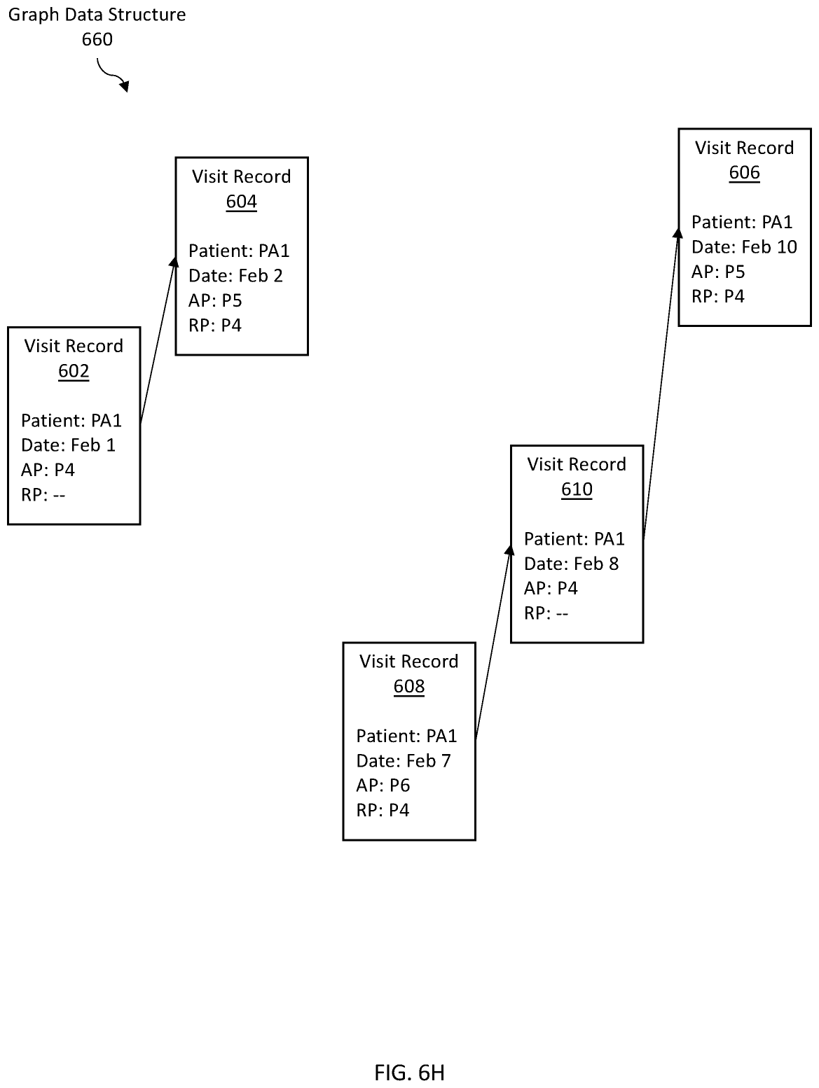

Graph data structures may be stored in one of a variety of commercially-available data abstractions. Examples include Apache Spark's DataFrames, and GraphFrames. A GraphFrame may include one or more vertex lists and one or more edge lists. Each vertex list or edge list may be stored as a DataFrame. Referring to FIG. 6E, for example, the graph illustrated in the figure may correspond to the following tables:

TABLE 1
Vertex List

| Index | Visit Record |
|---|---|
| a | Visit record 602 |
| b | Visit record 604 |
| c | Visit record 606 |
| d | Visit record 608 |
| e | Visit record 610 |

TABLE 2
Edge List

| Source | Destination |
|---|---|
| a | b |
| a | d |
| e | c |

An edge list may store additional information about edges in a graph. As an example, an edge list may store counts for each edge. Referring to FIG. 6F, for example, the graph illustrated in the figure may correspond to the following tables:

TABLE 3
Vertex List

| Index | Attending Physician (AP) |
|---|---|
| a | P4 |
| b | P5 |
| c | P6 |

TABLE 4
Edge List

| Source | Destination | Count |
|---|---|---|
| a | b | 2 |
| a | c | 1 |

A graph data structure may include one or more connected components. A connected component is a subgraph of a graph in which any two vertices are connected to each other by one or more paths. The vertices of a connected component are not connected to any additional vertices in the supergraph. Referring to FIG. 6E, for example, two connected components are within the graph. One connected component includes visit record 602 having paths to each of visit record 604 and visit record 608. Another connected component includes visit record 610 having a path to visit record 606.

A vertex list may store additional information about vertices in a graph. As an example, a vertex list may identify each connected component and respective vertices associated with each. Each connected component within a graph is assigned a connected component ID. The vertex list stores the connected component ID for each vertex. Referring again to FIG. 6E, for example, the edge list may include the connected component IDs as follows:

TABLE 5
Vertex List

| Index | Visit Record | Connected Component ID |
|---|---|---|
| a | Visit record 602 | 1 |
| b | Visit record 604 | 1 |
| c | Visit record 606 | 2 |
| d | Visit record 608 | 1 |
| e | Visit record 610 | 2 |

In one or more embodiments, a visit path graph data structure 108 stores information representing one or more patient visit paths (also referred to as a “visit path”). A visit path includes a series of patient visits that are related by an explicit referral and/or implicit referral. A visit path graph data structure 108 includes vertices 110 representing visits. Additionally, a visit path graph data structure 108 includes edges 112 representing an explicit referral and/or implicit referral between two visits. An edge 112 is a directed edge, such that the edge 112 indicates one visit is a referral source and another visit is a referral destination.

In one or more embodiments, a physician network graph data structure 114 stores information representing one or more physician referral networks (also referred to as a “physician network”). A physician network includes a series of physicians that are related by an explicit referral and/or implicit referral. A physician network graph data structure 114 includes vertices 116 representing attending physicians. Additionally, a physician network graph data structure 114 includes edges 118 representing an explicit referral and/or implicit referral between two physicians. An edge 118 is a directed edge, such that the edge 118 indicates one physician is a referring physician and another physician is a referee physician.

In one or more embodiments, a provider network graph data structure 120 stores information representing one or more provider referral networks (also referred to as a “provider network”). A provider network includes a series of healthcare providers and/or healthcare organizations (such as hospitals and/or medical groups) that are related by an explicit referral and/or implicit referral. A provider network graph data structure 120 includes vertices 122 representing providers associated with attending physicians. Additionally, a provider network graph data structure 120 includes edges 124 representing an explicit referral and/or implicit referral between two providers. An explicit referral between two providers is based on an explicit referral between two physicians corresponding respectively to the two providers. An implicit referral between two providers is based on an implicit referral between two physicians corresponding respectively to the two providers. An edge 124 is a directed edge, such that the edge 124 indicates one provider is a referring provider and another provider is a referee provider.

In an embodiment, a physician network and/or provider network is generated based on a single visit path. In another embodiment, a physician network and/or provider network is generated based on multiple visit paths. Any criteria may be used for identifying the multiple visit paths. As an example, a physician network may be generated based on visit paths corresponding to the same patient. As another example, a physician network may be generated based on visit paths including a visit within the last five years. As another example, a physician network may be generated based on visit paths including a visit during which a particular medical procedure was performed. As another example, a physician network may be generated based on visit paths including a visit within a particular geographical vicinity. Any combination of the above example criteria may also be used.

In one or more embodiments, graph data structures for additional and/or alternative paths and/or networks may be used. Any field within a visit record 106 may be used to generate a network. As described above, an attending physician field of visit records 106 may be used to generate a physician network. A healthcare provider field of visit records 106 may be used to generate a provider network. Additionally or alternatively, an insurance company field (insurance company(s) to which a claim was submitted for the visit) of visit records 106 may be used to generate a relational network on insurance companies. A diagnosis field of visit records 106 may be used to generate a relational network on diagnoses. A prescribed medication field of visit records 106 may be used to generate a relational network on prescribed medications. A medical procedure field of visit records 106 may be used to generate a relational network on medical procedures.

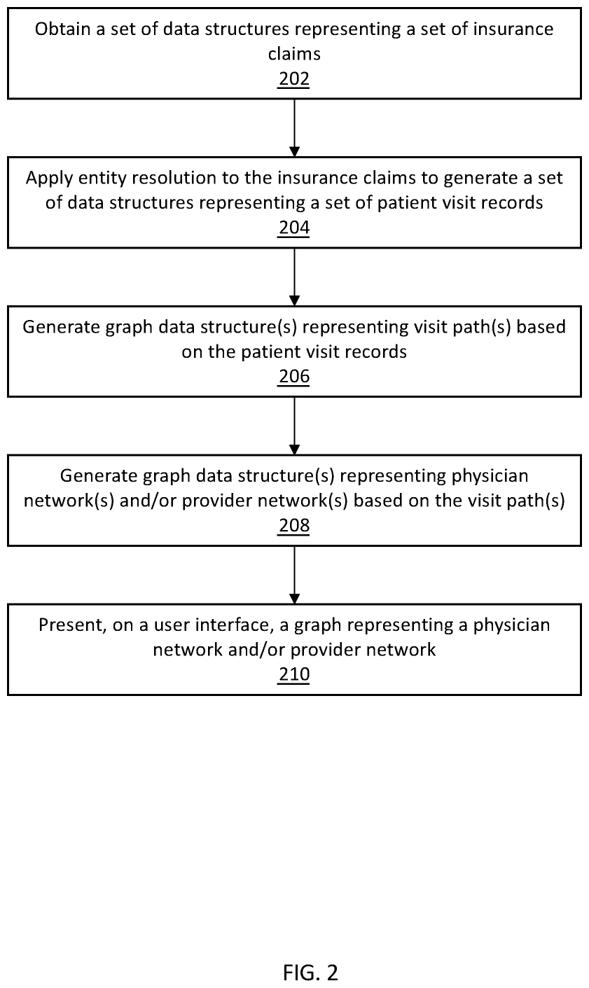

In one or more embodiments, a patient visit record generator 126 refers to hardware and/or software configured to perform operations described herein for generating a patient visit record 106 based on one or more insurance claim records 104. Examples of operations for generating a patient visit record 106 are described below with reference to FIG. 2.

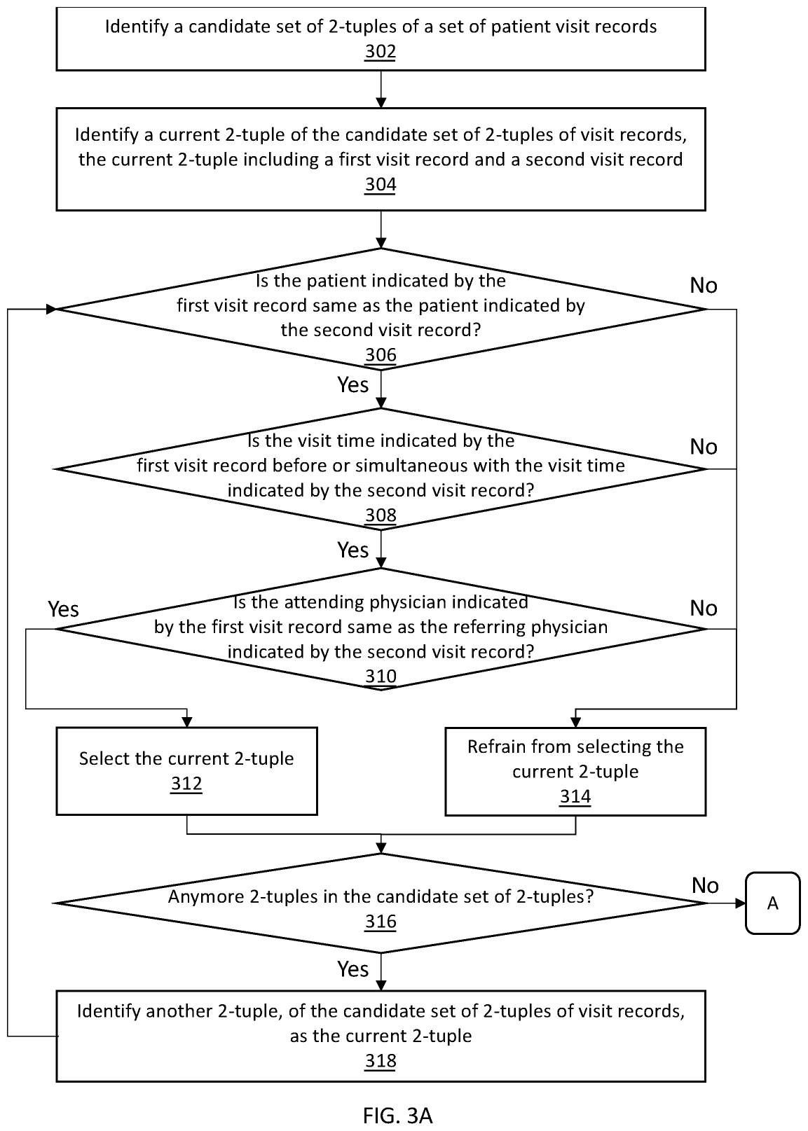

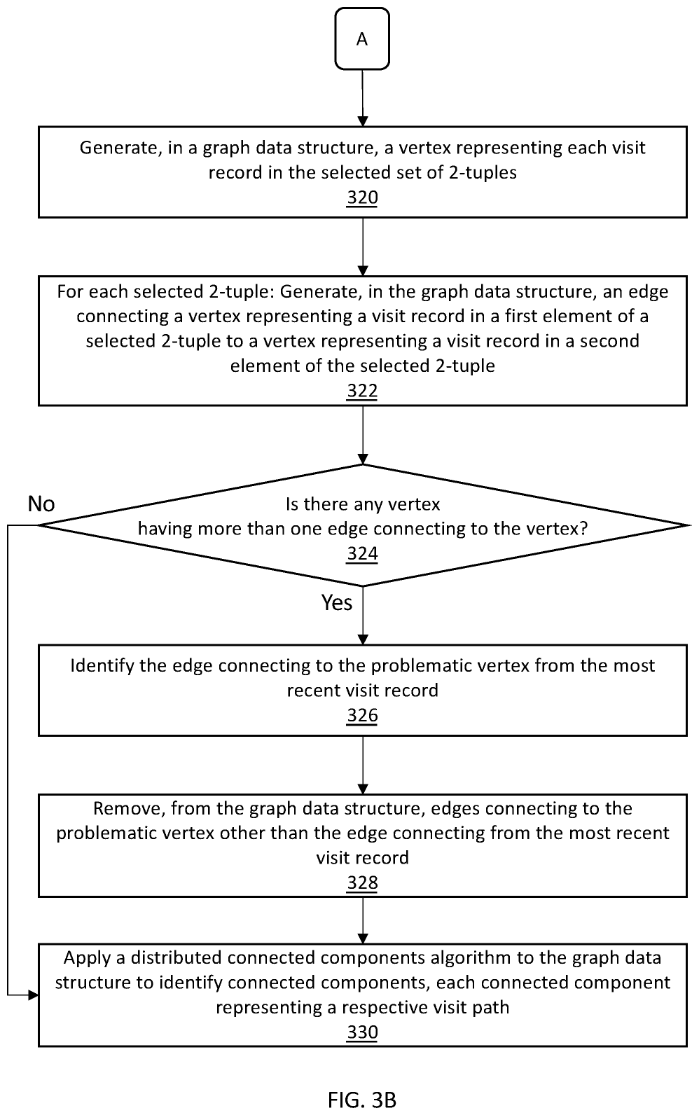

In one or more embodiments, a visit path generator 128 refers to hardware and/or software configured to perform operations described herein for generating a visit path graph data structure 108 based on one or more visit records 106. Examples of operations for generating a visit path graph data structure 108 are described below with reference to FIGS. 2, 3A-B, and 4A-B.

A visit path generator 128 is configured to generate one or more types of visit paths. As an example, one visit path type may include visit paths based on explicit referrals (also referred to as “referred visit paths”). Another visit path type may include visit paths based on implicit referrals (also referred to as “inferred visit paths”). Each visit path type is associated with a respective set of criteria. A visit path of a particular type links together visit records 106 that satisfy the criteria corresponding to the particular type. As illustrated, for example, criteria 130a correspond to visit path type 132a; criteria 130b correspond to visit path type 132b.

An aggregated visit path that combines visit paths of different types may be referred to as a “joint visit path.” As an example, a referred visit path may include Visit K→Visit N→Visit G. An implicit visit path may include Visit K→Visit N→Visit H. The two visit paths may be aggregated together to form a joint visit path. The joint visit path may include a branch at Visit N. Visit K is connected to Visit N. Then Visit N is connected to both Visit G and Visit H.

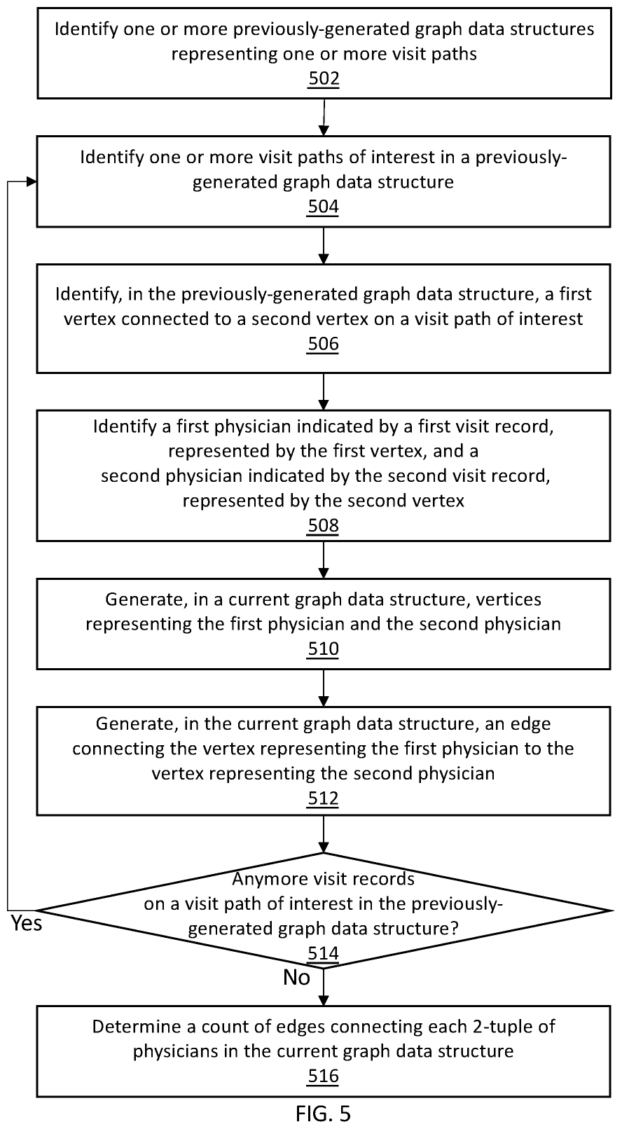

In one or more embodiments, a network generator 134 refers to hardware and/or software configured to perform operations described herein for generating a physician network graph data structure 114 and/or provider network graph data structure 120 based on one or more visit path graph data structures 108. Examples of operations for generating a physician network graph data structure 114 and/or provider network graph data structure 120 are described below with reference to FIGS. 2 and 5.

In an embodiment, a patient visit record generator 126, visit path generator 128, and/or network generator 134 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a mainframe, a television, a content receiver, a set-top box, a printer, a mobile handset, a smartphone, a personal digital assistant (PDA).

In an embodiment, a patient visit record generator 126, visit path generator 128, and/or network generator 134 operates in distributed function. Each of the patient visit record generator 126, visit path generator 128, and/or network generator 134 may include multiple computing processors, cores, and/or servers operating in parallel. Distributed processing is especially necessary where the data to be processed is voluminous. As an example, given a set of visit records, a candidate set of 2-tuples of visit records need to be analyzed for possible paths between two visit records (further details regarding identifying a candidate set of 2-tuples of visit records are described below with reference to Operation 302). Where there are 1,000 visit records, the candidate set of 2-tuples may include up to 999,000 2-tuples to be analyzed. Where there are 10,000 visit records, the candidate set of 2-tuples may include up to 99,990,000 2-tuples to be analyzed. Hence, distributed processing increases the efficiency in which paths and/or networks are determined.

In particular, a visit path generator 128 and/or network generator 134 may apply a distributed connected algorithm for determining connected components within a graph. An example of a distributed connected algorithm is implemented by the connectedComponents function in Apache Spark's GraphFrames application programming interface (API). The connectedComponents function accepts the following parameters: a connected component algorithm to be used; a checkpoint interval in terms of number of iterations; and a broadcast threshold in propagating component assignments. The connectedComponents function returns a DataFrame with a new vertices column for “Component.”

In one or more embodiments, a user interface 136 refers to hardware and/or software configured to facilitate communications between a user and a patient visit record generator 126, visit path generator 128, and/or network generator 134. A user interface 136 renders user interface elements that may present information and/or receive user input. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, a user interface 136 presents a visit path graph 138. A visit path graph 138 is a graphical presentation of a visit path graph data structure 108. A visit path graph 138 includes a set of nodes and a set of links connecting particular nodes. The nodes represent visits and the links represent paths between the visits.

In an embodiment, a user interface 136 presents a physician network graph 140 and/or provider network graph 142. A physician network graph 140 is a graphical presentation of a physician network graph data structure 114. A physician network graph 140 includes a set of nodes and a set of links connecting particular nodes. The nodes represent physicians and the links represent referrals between the physicians. Similarly, a provider network graph 142 is a graphical presentation of a provider network graph data structure 120. A provider network graph 142 includes a set of nodes and a set of links connecting particular nodes. The nodes represent providers and the links represent referrals between the providers.

Additionally or alternatively, a user interface 136 may present other graphs presenting other networks, paths, and/or relationships that are determined based on visit records 106.

Visualizations associated with a graph may be used to represent various information. As an example, a size of a node may represent a count of the referrals directed towards the node. As another example, a length of a link connecting two nodes that represent two physicians may represent a physical distance between the two physicians. As another example, a length of a link connecting two nodes that represent two physicians may be proportional to a count of edges, in a graph data structure representing a physician network, derived from visit paths of interest. As another example, a color of a node representing a physician may represent a provider associated with the physician.

A graph may be interactive. A node may be selectable for requesting additional information about the physician and/or provider represented by the node. A link may be selectable for requesting additional information about referrals between physicians and/or providers.

### 3. GENERATING AND PRESENTING GRAPH DATA STRUCTURES REPRESENTING PATIENT VISIT PATHS AND PHYSICIAN REFERRAL NETWORKS

FIG. 2 illustrates an example set of operations for generating and presenting patient visit paths and physician referral networks based on a set of insurance claims, in accordance with one or more embodiments. One or more operations illustrated in FIG. 2 may be modified, rearranged, or omitted all together. Accordingly, the particular sequence of operations illustrated in FIG. 2 should not be construed as limiting the scope of one or more embodiments.

One or more embodiments include obtaining a set of data structures representing a set of insurance claims (Operation 202). A system obtains a set of data structures representing a set of insurance claims from one or more data sources. Different types of data structures may be used. Different schemas may be used for storing the information. The system applies a pre-processing operation to standardize the insurance claims. The system may, for example, convert obtained insurance claim information to comply with the 837P data format. The system may, for example, store the 837P formatted insurance claim information in a columnar data storage format.

One or more embodiments include applying entity resolution (ER) to the insurance claims to generate a set of data structures representing a set of patient visit records (Operation 204). The system applies ER to the insurance claims to identify groups of insurance claims referring to the same patient visit. Various ER algorithms may be used. In an embodiment, the system aggregates insurance claims based on (a) time and (b) actors (such as patients and physicians). Insurance claims within a particular time window and with matching actors are aggregated into a single visit. In another embodiment, the system applies ER and uses likelihood and probability scoring to determine which insurance claims refer to the same patient visit.

As an example, a set of insurance claims may include the following information:

TABLE 6
Example Insurance Claim Records

| Insurance Claim | Visit | Patient | Medical |
|---|---|---|---|
| Record ID | Date | Address | Diagnosis |
| 1 | Mar. 1, 2019 | 4 Main St, | Astigmatism |
| | | Concord, MA | |
| 2 | Feb. 15, 2019 | Concord, MA | Myopia |
| 3 | Mar. 1, 2019 | Concord, MA | Ocular |
| | | | Hypertension |
| 4 | Feb. 20, 2019 | 62 4th Ave, | Cataract |
| | | Boston, MA | |

A system may apply ER to the insurance claim records. The system may perform pairwise comparison of the insurance claim records. The system may discard pairs including the same insurance claim records in different orders. Record ID 1 is compared with Record IDs 2, 3, 4. Record ID 2 is compared with Record IDs 3, 4. Record ID 3 is compared with Record ID 4.

In comparing Record ID 1 and Record ID 2, the system may compare the visit dates. The system may determine that the visit dates do not match. The system may compare the patient addresses. The system may determine a high similarity score for the patient address field. The system may compare the medical diagnoses. The system may determine a low similarity score for the diagnosis field. The system may obtain an intermediate overall score for the pair Record ID 1 and Record ID 2.

In comparing Record ID 1 and Record ID 3, the system may compare the visit dates. The system may determine that the visit dates do match. Further, the system may determine a high similarity score for the patient address field. The system may determine a low similarity score for the medical diagnosis field. The system may obtain a high overall score for the pair Record ID 1 and Record ID 3.

In comparing Record ID 1 and Record ID 4, the system may compare the visit dates. The system may determine that the visit dates do not match. Further, the system may determine a low similarity score for the patient address field. The system may determine a low similarity score for the medical diagnosis field. The system may obtain a low overall score for the pair Record ID 1 and Record ID 4.

The system may compare each overall score for each pair of insurance claim records to a threshold value. The system may determine that the overall score for the pair Record ID 1 and Record ID 3 is above the threshold value. The system may determine that the overall scores for all other pairs are below the threshold value. The system may determine that Record ID 1 and Record ID 3 refer to the same patient visit; Record ID 2 refers to another patient visit; Record ID 4 refers to another patient visit.

Based on a group of insurance claims referring to the same patient visit, the system aggregates the information and compiles a patient visit record. Where a same field is included in two insurance claim records, the system may merge, concatenate, append, or otherwise compile the respective information in the field from the two insurance claim records into a visit record. Where a particular field is included in one insurance claim record, and the field is not included in another insurance claim record, the system may include the available information in the field into a visit record.

Referring back to the example insurance claim records shown in Table 6 above, Record ID 1 and Record ID 3 include different names for the patient. The system may include both names into the visit record. Record ID 1 and Record ID 3 include different addresses for the patient, wherein “4 Main St, Concord, Mass.” is geographically within “Concord, Mass.” The system may include “4 Main St, Concord, Mass.” into the visit record, without including “Concord, Mass.” Hence, the visit records compiled from Record IDs 1-4 may include the following information:

TABLE 7
Example Patient Visit Records

| | Related | | | |
|---|---|---|---|---|
| Patient | Insurance | | | |
| Visit | Claim | Visit | | Medical |
| Record ID | Record ID(s) | Date | Address | Diagnosis |
| 1 | 1, 3 | Mar. 1, 2019 | 4 Main St, | Astigmatism, |
| | | | Concord, MA | Ocular |
| | | | | Hypertension |
| 2 | 2 | Feb. 15, 2019 | Concord, MA | Myopia |
| 3 | 4 | Feb. 20, 2019 | 62 4th Ave, | Cataract |
| | | | Boston, MA | |

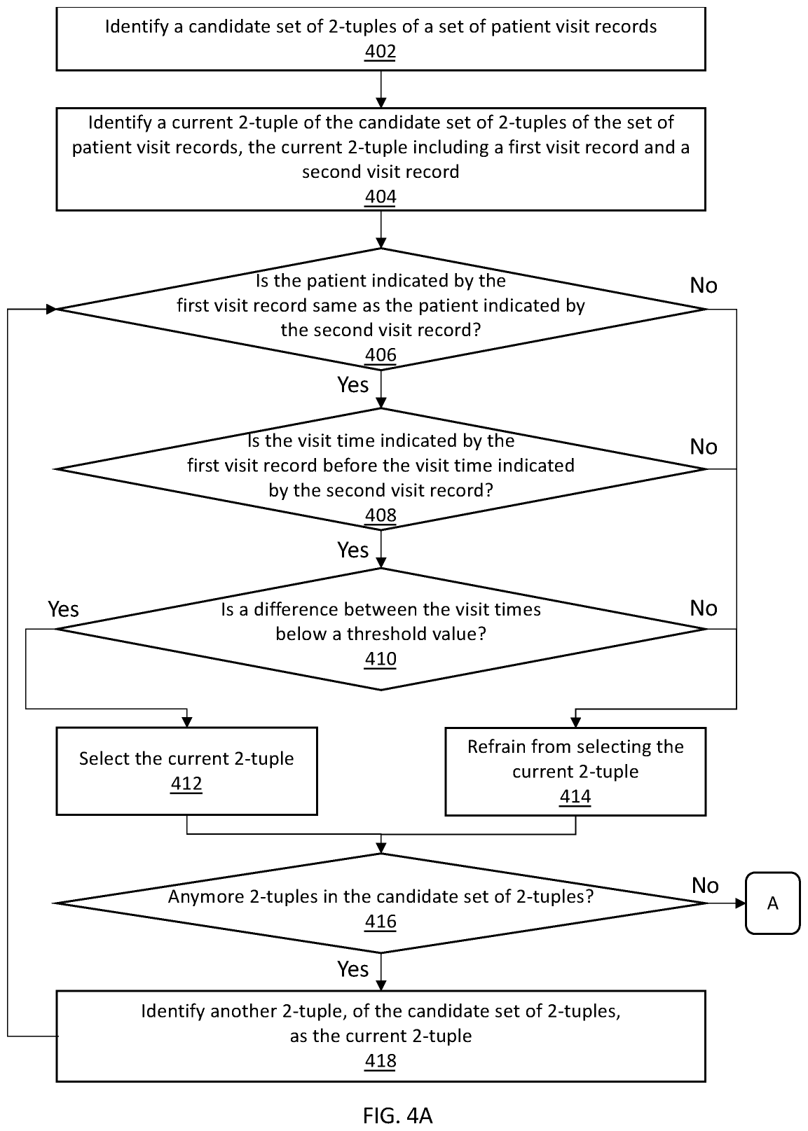

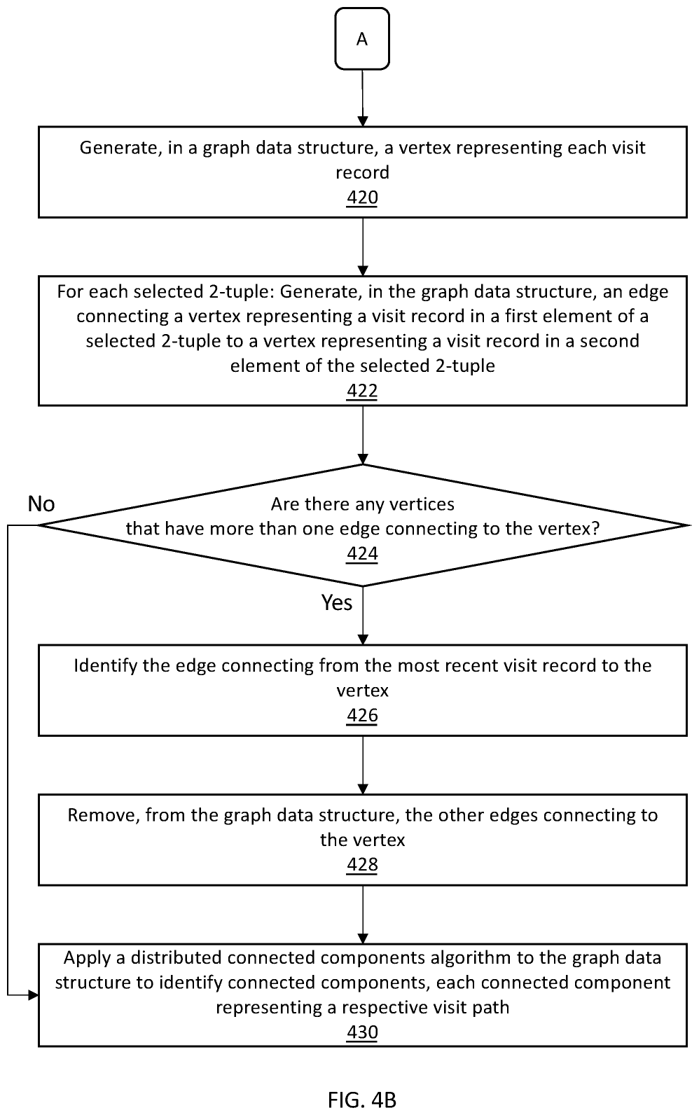

One or more embodiments include generating one or more graph data structures representing one or more visit paths based on the patient visit records (Operation 206). The system generates one or more graph data structures representing one or more visit paths based on the patient visit records. The system performs pairwise comparison on each 2-tuple of visit records. A 2-tuple includes two elements, each element corresponding to an object. The ordering of the two objects is significant. Hence, the 2-tuple (A, B) is different from the 2-tuple (B, A). In the 2-tuple (A, B), A is referred to as corresponding to the first element of the 2-tuple, and B is referred to as corresponding to the second element of the 2-tuple. The system determines whether each 2-tuple satisfies a set of criteria associated with a visit path type. If the 2-tuple satisfies the criteria, then the system selects the 2-tuple as being included in a visit path of the visit path type. If the 2-tuple does not satisfy the criteria, then the system refrains from selecting the 2-tuple as being included in any visit path of the visit path type. Examples of operations for generating graph data structures representing visit paths are described below with reference to FIGS. 3A-B and FIGS. 4A-B.

One or more embodiments include generating one or more graph data structures representing one or more physician networks and/or provider networks based on one or more visit paths (Operation 208). The system generates one or more graph data structures representing one or more physician networks and/or provider networks and/or other networks, based on one or more visit paths. The system identifies adjacent visits on a visit path. The system identifies the values indicated by the adjacent visits for a particular field (such as the physician field, or the provider field, or any other field). The system generates an edge connecting the identified values (such as connecting two physicians, or connecting two providers, or connecting two diagnosis, or connecting two medical procedures). Examples of operations for generating one or more graph data structures representing one or more physician networks and/or provider networks, based on one or more visit paths, are described below with reference to FIG. 5.

In an embodiment, the system generates one or more graph data structures representing one or more physician networks and/or provider networks based on referred visit paths, without using any inferred visit paths. In an alternative embodiment, the system generates one or more graph data structures representing one or more physician networks and/or provider networks based on inferred visit paths, without using any referred visit paths. In an alternative embodiment, the system generates one or more graph data structures representing one or more physician networks and/or provider networks based on joint visit paths.

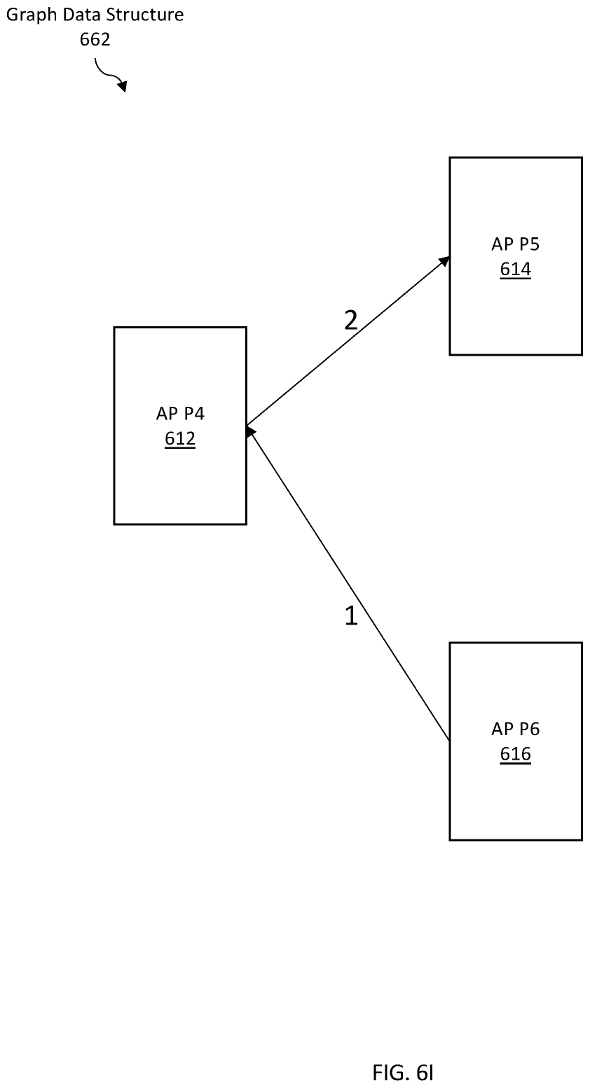

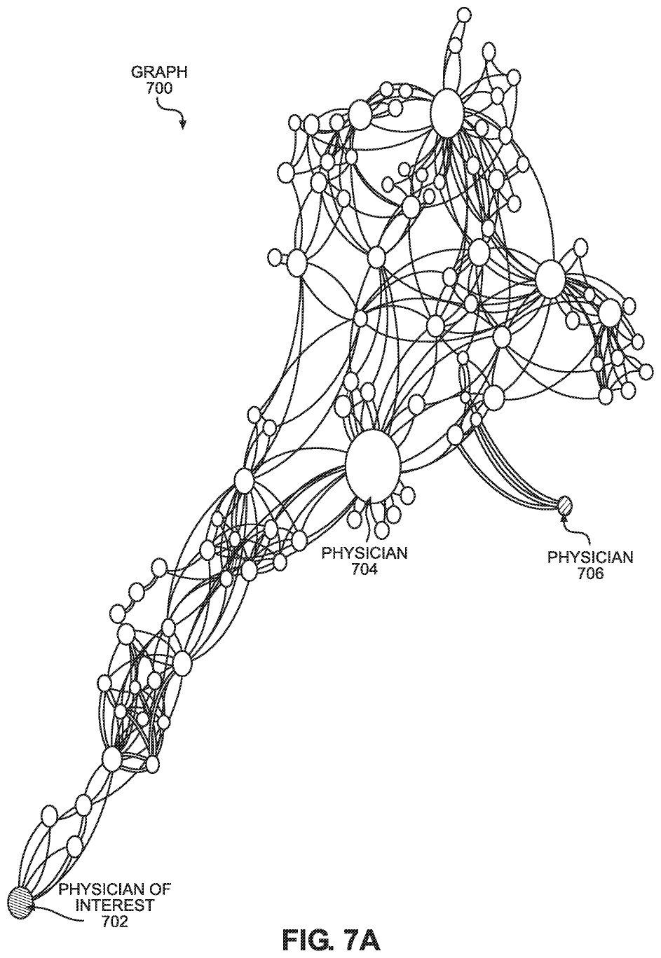

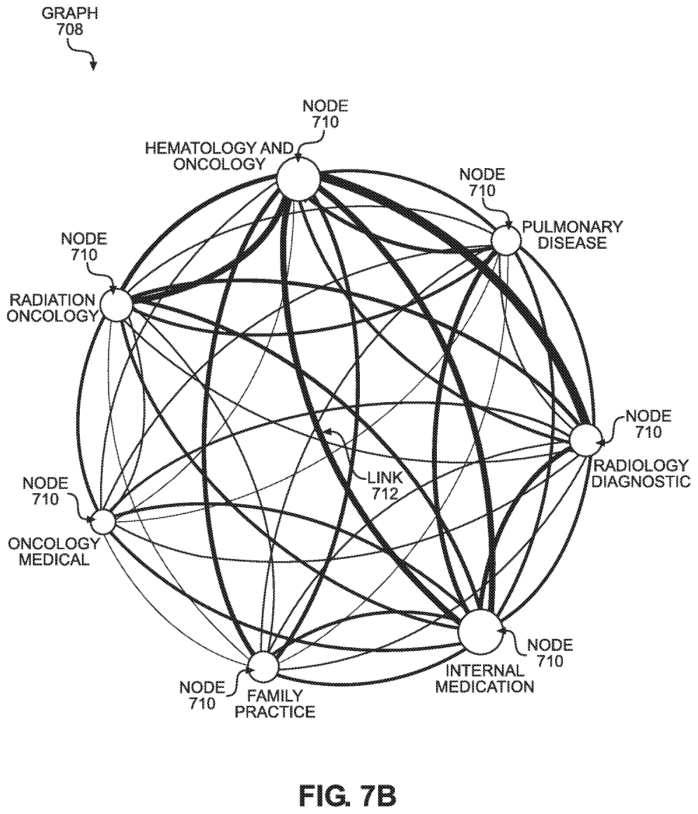

One or more embodiments include presenting, on a user interface, a graph representing a physician network and/or provider network (Operation 210). The system presents, on a user interface, a graph representing a physician network and/or provider network. The user interface shows a set of nodes and a set of links connecting certain nodes. Each node represents a physician and/or provider. The graph representing a physician network and/or provider network is presented based on a graph data structure representing a physician network and/or provider network, generated at Operation 208. An example of a user interface presenting a graph of a physician network is described below with reference to FIG. 7A. An example of a user interface presenting a graph of a relational network on medical departments is described below with reference to FIG. 7B.

In an embodiment, the system concurrently presents, on a user interface, (a) a physician and/or provider network determined based on explicit referrals (without using any implicit referrals), and (b) a physician and/or provider network determined based on implicit referrals (without using any explicit referrals). The user interface presents the physician and/or provider network determined based on explicit referrals using one visualization (such as, a particular color of nodes and/or links). The user interface presents the physician and/or provider network determined based on implicit referrals using another visualization (such as, a different color of nodes and/or links). In an embodiment, the system may present options to a user for selecting a display view. The options include: (a) viewing a physician and/or provider network determined based on explicit referrals only, (b) viewing a physician and/or provider network determined based on implicit referrals only, or (c) concurrently viewing both a physician and/or provider network determined based on explicit referrals and a physician and/or provider network determined based on implicit referrals.

In an alternative embodiment, the system presents, on the user interface, a graph representing one or more visit paths. The user interface shows a set of nodes and a set of links connecting certain nodes. Each node represents a visit path. The graph representing one or more visit paths is presented based on a graph data structure representing one or more visit paths, generated at Operation 206.

FIGS. 3A-B illustrate an example set of operations for generating a graph data structure representing a visit path based on explicit referrals, in accordance with one or more embodiments. One or more operations illustrated in FIGS. 3A-B may be modified, rearranged, or omitted all together. Accordingly, the particular sequence of operations illustrated in FIGS. 3A-B should not be construed as limiting the scope of one or more embodiments.

One or more embodiments include identifying a candidate set of 2-tuples of a set of patient visit records (Operation 302). A system identifies a set of patient visit records. Various ways of identifying 2-tuples may be used.

In an embodiment, the system identifies all possible 2-tuples of visit records from the set of visit records as the candidate set of 2-tuples of visit records. Given a set of n visit records, then the number of possible 2-tuples is n×(n−1). Hence, the number of 2-tuples in the candidate set of 2-tuples is n×(n−1).

In an alternative embodiment, the system identifies groups of visit records that share the same patient. The system identifies all possible 2-tuples within each group of visit records. Comparing only visit records referencing the same patient (rather than comparing all visit records) reduces the number of 2-tuples to be analyzed.

One or more embodiments include identifying a current 2-tuple of the candidate set of 2-tuples of visit records (Operation 304). The system iterates through the candidate set of 2-tuples to analyze each one. Initially, the system identifies any of the candidate set of 2-tuples to analyze first. Thereafter, the system identifies any 2-tuple that has not yet been iterated for analysis. The identified 2-tuple may be referred to as the “current 2-tuple.” The visit record corresponding to the first element of the current 2-tuple may be referred to as a “first visit record.” The visit record corresponding to the second element of the current 2-tuple may be referred to as a “second visit record.”

One or more embodiments include determining whether the patient indicated by the first visit record is the same as the patient indicated by the second visit record (Operation 306). One criteria for selecting the current 2-tuple as being part of a visit path is matching patients. The system identifies the patient indicated by the first visit record. The system identifies the patient indicated by the second visit record. The system determines whether the two patients are the same.

One or more embodiments include determining whether the visit time indicated by the first visit record is before or simultaneous with the visit time indicated by the second visit record (Operation 308). Another criteria for selecting the current 2-tuple as being part of a visit path is matching visit times. The system identifies the visit time indicated by the first visit record. The system identifies the visit time indicated by the second visit record. The system determines whether the visit time indicated by the first visit record is before or simultaneous with the visit time indicated by the second visit record. The term “simultaneous” refers to a first visit time being within the same time unit as a second visit time. The time unit may be, for example, a day, or an hour, or a week. As an example, where the time unit is a day, a visit time of September 1 at 10 am and another visit time of September 1 at 3 pm may be referred to as being “simultaneous.” A visit time of September 1 at 5 pm may be referred to as being “before” a visit time of September 2 at 9 am.

One or more embodiments include determining whether the attending physician indicated by the first visit record is the same as the referring physician indicated by the second visit record (Operation 310). Another criteria for selecting the current 2-tuple as being part of a visit path is matching physicians. The system identifies the attending physician time indicated by the first visit record. The system identifies the referring physician indicated by the second visit record. The system determines whether the attending physician indicated by the first visit record is the same as the referring physician indicated by the second visit record.

If all criteria are satisfied, then one or more embodiments include selecting the current 2-tuple (Operation 312).

If any of the criteria are not satisfied, then one or more embodiments include refraining from selecting the current 2-tuple (Operation 314).

In alternative embodiments, fewer, additional, and/or alternative criteria (than the criteria described with reference to Operations 306-310) may be used in determining whether to select the current 2-tuple as being part of a visit path.

One or more embodiments include determining whether there are any additional 2-tuples in the candidate set of 2-tuples to be processed (Operation 316). The system determines whether there are any 2-tuples, in the candidate set of 2-tuples, that has not yet been iterated.

If there is at least one 2-tuple, in the candidate set of 2-tuples, that has not yet been iterated, then one or more embodiments include identifying another 2-tuple, of the candidate set of 2-tuples of visit records, as the current 2-tuple (Operation 318). The system identifies a 2-tuple that has not been iterated as the “current 2-tuple.” The system then iterates Operations 306-316 with respect to the current 2-tuple.

The system iterates Operation 306-316 until each of the candidate set of 2-tuples has been iterated.

If there are no more 2-tuples in the candidate set of 2-tuples to be processed, then one or more embodiments include generating, in a graph data structure, a vertex representing each visit record in the selected set of 2-tuples (Operation 320). The system generates a vertex list. The system creates entries in the vertex list. Each entry stores an identifier corresponding to a respective visit record in the selected set of 2-tuples. There is not necessarily any entry corresponding to a visit record not within the selected set of 2-tuples.

As an example, a candidate set of 2-tuples includes:

### Visit Record ID 1-Visit Record ID 2;

### Visit Record ID 1-Visit Record ID 3;

### Visit Record ID 1-Visit Record ID 4;

### Visit Record ID 2-Visit Record ID 1;

### Visit Record ID 2-Visit Record ID 3;

### Visit Record ID 2-Visit Record ID 4;

### Visit Record ID 3-Visit Record ID 1;

### Visit Record ID 3-Visit Record ID 2;

### Visit Record ID 3-Visit Record ID 4;

### Visit Record ID 4-Visit Record ID 1;

### Visit Record ID 4-Visit Record ID 2;

### Visit Record ID 4-Visit Record ID 3.

A system may determine that only the following 2-tuples satisfy a set of criteria for selection: Visit Record ID 1-Visit Record ID 3; Visit Record ID 2-Visit Record ID 3. Hence the selected set of 2-tuples may be: Visit Record ID 1-Visit Record ID 3; Visit Record ID 2-Visit Record ID 3.

The system may determine that the visit records included in the selected set of 2-tuples are: Visit Record ID 1; Visit Record ID 2; and Visit Record ID 3. Visit Record ID 4 is not a visit record in the selected set of 2-tuples. The system may generate a vertex list including three entries. The first entry corresponds to a vertex representing Visit Record ID 1. The second entry corresponds to a vertex representing Visit Record ID 2. The third entry corresponds to a vertex representing Visit Record ID 3. There is not necessarily any vertex representing Visit Record ID 4.

One or more embodiments include generating, in the graph data structure, an edge connecting a vertex representing a visit record in a first element of a selected 2-tuple to a vertex representing a visit record in a second element of the selected 2-tuple (Operation 322).

The system generates an edge list. The system creates entries in the edge list, one entry for each selected 2-tuple. Each entry corresponds to an edge. Each entry stores two indices into the vertex list: one index into the vertex list corresponds to a vertex at the head or source of an edge; the other index into the vertex list corresponds to a vertex at the tail or destination of the edge.

Initially the system identifies any of the selected set of 2-tuples to analyze first. The system identifies a first visit record in a first element of the current selected 2-tuple. The system identifies a second visit record in a second element of the current selected 2-tuple. The system identifies an index into the vertex list corresponding to the first visit record. The system stores the index as a “source” in a particular entry of the edge list. The system identifies an index into the vertex list corresponding to the second visit record. The system stores the index as a “destination” in the particular entry of the edge list.

Thereafter the system identifies another selected 2-tuple, which has not yet been iterated, for analysis. The system iterates the above process to store a “source” and “destination” for another edge. The system iterates the above process until each of the selected set of 2-tuples has been analyzed.

One or more embodiments include determining whether there is any vertex having more than one edge connecting to the vertex (Operation 324). Since the system is designed to identify one visit record that leads to a particular visit record, the system attempts to determine visit paths in which only one vertex connects to each vertex. Hence, each vertex to which more than one edge connects may be referred to as “problematic vertex.” Meanwhile, the system allows a particular vertex to connect to one or more vertices.

The system searches through the “destination” column of the edge list. The system identifies any vertex that has been included more than one time in the “destination” column. Any vertex included more than one time in the “destination” column is a vertex to which more than one edge connects, and is thereby a “problematic vertex.”

One or more embodiments include identifying the edge connecting to the problematic vertex from the most recent visit record (Operation 326). The system identifies each edge in which the problematic vertex is a destination. The system identifies each vertex in the source of the identified edges. The system identifies each visit record corresponding to the identified vertices. The system identifies each visit time indicated by the identified visit records. The system identifies a most recent visit time from the identified visit times. The visit record corresponding to the identified most recent visit time may be referred to as a “most recent visit record.” The system thereby identifies the edge that connects the vertex representing the most recent visit record to the problematic vertex.

One or more embodiments include removing, from the graph data structure, edges connecting to the problematic vertex other than the edge connecting from the most recent visit record (Operation 328). The system removes, from the graph data structure, all edges connecting to the problematic vertex other than the edge connecting from the most recent visit record. The system removes and/or deletes the edges from the edge list.

After removal of the unwanted edges, the graph data structure represents one or more visit paths, as determined by the system.

One or more embodiments include applying a distributed connected components algorithm to the graph data structure to identify connected components, each connected component representing a respective visit path (Operation 330). The system applies a distributed connected components algorithm to the graph data structure to identify connected components. Due to the voluminous data that may be associated with the graph data structure, the system uses a distributed connected components algorithm to efficiently process the information. The system thereby identifies the connected components within the graph data structure. Each connected component represents a respective visit path.

Referring to FIG. 6H, for example, a system may apply a connected components algorithm to graph data structure 660. Based on the connected components algorithm, the system may identify one connected component as including visit record 602→visit record 604. The connected component represents one visit path. The system may identify another connected component as including visit record 608→visit record 610→visit record 606. The connected component represents a separate visit path.

FIGS. 4A-B illustrate an example set of operations for generating a graph data structure representing a visit path based on implicit referrals, in accordance with one or more embodiments. One or more operations illustrated in FIGS. 4A-B may be modified, rearranged, or omitted all together. Accordingly, the particular sequence of operations illustrated in FIGS. 4A-B should not be construed as limiting the scope of one or more embodiments.

Operations of FIGS. 3A-B and FIGS. 4A-B are similar, except that different criteria for selecting 2-tuples, from the candidate set of 2-tuples of visit records, are used. Criteria for determining a referred visit path are described above with reference to Operations 306-310 provide the set of criteria to be used. Criteria for determining an inferred visit path are described below.

One or more embodiments include determining whether the patient indicated by the first visit record is the same as the patient indicated by the second visit record (Operation 406). One criteria for selecting the current 2-tuple as being part of a visit path is matching patients. The system identifies the patient indicated by the first visit record. The system identifies the patient indicated by the second visit record. The system determines whether the two patients are the same.

One or more embodiments include determining whether the visit time indicated by the first visit record is before the visit time indicated by the second visit record (Operation 408). Another criteria for selecting the current 2-tuple as being part of a visit path is matching visit times. The system identifies the visit time indicated by the first visit record. The system identifies the visit time indicated by the second visit record. The system determines whether the visit time indicated by the first visit record is before the visit time indicated by the second visit record.

One or more embodiments include determining whether a difference between the visit times, of the first visit record and the second visit record, is below a threshold value (Operation 410). Another criteria for selecting the current 2-tuple as being part of a visit path is closeness in time. The system determines whether a difference between the visit times, of the first visit record and the second visit record, is below a threshold value.

In alternative embodiments, fewer, additional, and/or alternative criteria (than the criteria described with reference to Operations 406-410) may be used in determining whether to select the current 2-tuple as being part of a visit path.

Reviewing the criteria for determining a referred visit path and an inferred visit path, one criteria may be laxer for a referred visit path than for an inferred visit path, while another criteria may be stricter for the referred visit path than for the inferred visit path. First, the criteria described in Operations 306 and 406 are the same. Second, the criteria described in Operation 308 is laxer than the criteria described in Operation 408. The criteria associated with Operation 308 allows “before or simultaneous,” whereas the criteria associated with Operation 408 requires “before” only. Third, the criteria described in Operation 308 is stricter than the criteria described in Operation 408. The criteria described in Operation 308 requires an explicit referral to link two visits. An attending physician of the first visit must have officially submitted an explicit referral into a medical system. At least one insurance claim of the first visit and/or the second visit must reflect the explicit referral. No such criteria is required in determining inferred visit paths. A visit path may be determined, even where there is no explicit referral. The criteria described in Operation 408 merely requires that the time difference between two visits be within a certain threshold range.

FIG. 5 illustrates an example set of operations for generating a graph data structure representing a physician referral network, in accordance with one or more embodiments. One or more operations illustrated in FIG. 5 may be modified, rearranged, or omitted all together. Accordingly, the particular sequence of operations illustrated in FIG. 5 should not be construed as limiting the scope of one or more embodiments.

Operations similar to those illustrated in FIG. 5 may be used to generate a graph data structure representing other relational networks, such as a relational network on providers, a relational network on medical diagnoses, a relational network on insurance companies, a relational network on prescribed medications, and a relational network on medical departments (such as family medicine, internal medicine, radiology, oncology, pulmonary disease).

One or more embodiments include identifying one or more previously-generated graph data structures representing one or more visit paths (Operation 502). A system identifies a previously-generated graph data structure representing one or more visit paths.

The system identifies a graph data structure representing one or more referred visit paths. Examples of operations for generating a graph data structure representing one or more referred visit paths are described above with reference to FIGS. 3A-B. As described above, each referred visit path may be associated with a connected component ID.

Additionally or alternatively, the system identifies a graph data structure representing one or more inferred visit paths. Examples of operations for generating a graph data structure representing one or more inferred visit paths are described above with reference to FIGS. 4A-B. As described above, each inferred visit path may be associated with a connected component ID.

Additionally or alternatively, the system generates a graph data structure representing one or more joint visit paths, based on (a) a graph data structure representing one or more referred visit paths and (b) a graph data structure representing one or more inferred visit paths. The system aggregates all referred visit paths and inferred visit paths to generate the graph data structure representing joint visit paths. The system may apply a connected components algorithm to the graph data structure representing joint visit paths to determine each separate joint visit path in the graph data structure. Each joint visit path may be associated with a connected component ID.

In an embodiment, the system may identify one or more graph data structures representing visit paths associated with a same particular patient. In an alternative embodiment, the system may identify graph data structures representing visit paths associated with multiple different patients.

One or more embodiments include identifying one or more visit paths of interest in a previously-generated graph data structure (FIG. 504). The system identifies one or more visit paths of interest in the graph data structure representing referred visit paths, the graph data structure representing inferred visit paths, and/or the graph data structure representing joint visit paths. The system may use connected component IDs to reference the visit paths of interest.

The system identifies a visit path of interest based on user input, a function, and/or an application. The user input, function, and/or application may specify one or more criteria for visit paths of interest. As an example, a user may specify a connected component ID corresponding to a visit path of interest. A system may receive the user-specified connected component ID.

As another example, a user may specify that only visit paths including a particular medical procedure constitute visit paths of interest. The user may specify a procedure code of the medical procedure. The system may receive the user-specified procedure code. The system may conduct a search of visit records to determine a subset of visit records including the user-specified procedure code. The system may identify all visit paths including any of the subset of visit records.

As another example, an application may select a random set of patients as patients of interest. The application may provide the patient IDs of the patients of interest to a system. The system may conduct a search of visit records to determine a subset of visit records including the patient IDs. The system may identify all visit paths including any of the subset of visit records.

One or more embodiments include identifying, in the previously-generated graph data structure, a first vertex connected to a second vertex on a visit path of interest (FIG. 506).

The system identifies a vertex list associated with the previously-generated graph data structure. The system identifies vertices associated with a visit path of interest. As an example, a system may identify a connected component ID associated with a visit path of interest. The system may search the vertex list for vertices associated with the connected component ID.

The system identifies any of the vertices associated with a visit path of interest for analysis. The system uses the identified vertex to index into the edge list. Based on the edge list, the system identifies another vertex connected to or connected from the identified vertex. The two vertices may be referred to as a “first vertex” and a “second vertex,” wherein the first vertex is connected to the second vertex.

One or more embodiments include identifying a first physician indicated by a first visit record, represented by the first vertex, and a second physician indicated by a second visit record, represented by the second vertex (FIG. 508). The system determines that the first vertex corresponds to a first visit record, and the second vertex corresponds to a second visit record. The system obtains the first visit record and the second visit record. The system identifies a first physician indicated by the first visit record. The system identifies a second physician indicated by the second visit record.

One or more embodiments include generating, in a current graph data structure, vertices representing the first physician and the second physician (FIG. 510). The system generates a current graph data structure that would represent a physician network. The system generates a vertex list for the current graph data structure.

The system stores a physician ID of the first physician into an entry of the vertex list. The system stores a physician ID of the second physician into another entry of the vertex list.

One or more embodiments include generating, in the current graph data structure, an edge connecting a vertex representing the first physician to a vertex representing the second physician (FIG. 512). The system generates an edge list for the current graph data structure.

The system stores the vertex corresponding to the first physician as a “source” in a particular entry of the edge list. The system stores the vertex corresponding to the second physician as a “destination” in the particular entry of the edge list.

One or more embodiments include determining whether there are any additional visit records on a visit path of interest, in the previously-generated graph data structure, that has not yet been processed (FIG. 514). The system determines whether there are any additional visit records on a visit path of interest, in the previously-generated graph data structure, that has not yet been processed. The system thereby ensures that each visit record on any visit path of interest, in any of the previously-generated graph data structures identified at Operation 502, are processed.

If there is at least one visit record on a visit path of interest that has not yet been iterated, the one or more embodiments include iterating Operations 504-514 with respect to a visit record that has not yet been iterated. The system iterates Operations 504-514 until each vertex on any visit path of interest has been iterated.

If there are no more visit records on any visit path of interest to be processed, then one or more embodiments include determining a count of edges connecting each 2-tuple of physicians in the current graph data structure (FIG. 516). The system searches through the edge list to identify any entries that refer to the same “source” physician and the same “destination” physician. For each 2-tuple of physicians, the number of entries corresponding to the 2-tuple is determined. The system may generate an aggregated edge list. The aggregated edge list includes each 2-tuple of physicians only once. The aggregated edge list includes an additional column for the count of edges, in the original edge list, associated with the same 2-tuple of physicians.

As an example, an edge list of a graph data structure representing a physician network may including the following information:

TABLE 8
Edge List

| Source | Destination |
|---|---|
| a | b |
| a | d |
| e | c |
| a | b |

A system may determine that the first entry and the last entry correspond to the same 2-tuple of vertices (a, b). The system may determine that the number of entries corresponding to (a, b) is two, the number of entries corresponding to (a, d) is one, and the number of entries corresponding to (e, c) is one. The system may generate an aggregated edge list, as follows:

TABLE 9
Aggregated Edge List

| Source | Destination | Count |
|---|---|---|
| a | b | 2 |
| a | d | 1 |
| e | c | 1 |

As shown above, the aggregated edge list includes each 2-tuple of vertices only once. Further the aggregated edge list includes an additional column for indicating the count of each 2-tuple in the original edge list.

In an alternative embodiment, the system may directly generate the aggregated edge list. At Operation 512, the system searches through the edge list to determine whether the edge that would be added to the edge list already exists. If so, the system increases a count associated with the edge, rather than creating a new entry in the edge list for the edge.

In one or more embodiments, the operations of FIG. 5 may be executed with respect to any field of a visit record. As illustrated, the operations of FIG. 5 are executed with respect to the physician field of a visit record. However, the operations of FIG. 5 may additionally or alternatively be executed with respect to the provider field, the insurance company field, the diagnosis field, the medical procedure field, the prescribed medication field, or any other field of a visit record. For example, where the operations of FIG. 5 are executed with respect to the provider field, then a graph data structure representing a provider network is generated.

### 4. EXAMPLE EMBODIMENT

Detailed examples are described below for purposes of clarity. Components and/or operations described below should be understood as specific examples which may not be applicable to certain embodiments. Accordingly, components and/or operations described below should not be construed as limiting the scope of any of the claims.

FIGS. 6A-I illustrate an example for generating patient visit paths and physician referral network, in accordance with one or more embodiments.

Referring to FIG. 6A, a set of visit records 650 are shown. Visit record 602 indicates that the patient ID is PA1, the visit date is February 1, the attending physician ID is P4, and the referring physician ID is blank. Visit record 604 indicates that the patient ID is PA1, the visit date is February 2, the attending physician ID is P5, and the referring physician ID is P4. Visit record 606 indicates that the patient ID is PA1, the visit date is February 10, the attending physician ID is P5, and the referring physician ID is P4. Visit record 608 indicates that the patient ID is PA1, the visit date is February 7, the attending physician ID is P6, and the referring physician ID is P4. Visit record 610 indicates that the patient ID is PA1, the visit date is February 8, the attending physician ID is P4, and the referring physician ID is blank.

Referring to FIG. 6B, a candidate set of 2-tuples of visit records 652 are shown. A system derives all possible 2-tuples from the visit records 650. The possible 2-tuples is the candidate set of 2-tuples 652. The number of 2-tuples in the candidate set of 2-tuples is n×(n−1)=5×4=20, where n is the number of visit records 650.

Referring to FIG. 6C, a selected set of 2-tuples of visit records 654 are shown. The system identifies a set of criteria for determining a visit path based on explicit referrals. The criteria include: (a) the patient indicated by a visit record in a first element of a 2-tuple is the same as the patient indicated by a visit record in a second element of the 2-tuple; (b) the visit time indicated by the visit record in the first element of the 2-tuple is before or simultaneous with the visit time indicated by the visit record in the second element of the 2-tuple; and (c) the attending physician indicated by the visit record in the first element of the 2-tuple is the same as the referring physician indicated by the visit record in the second element of the 2-tuple. The system applies the criteria to each of the candidate set of 2-tuples 652. The system determines that only four 2-tuples match the criteria: visit record 602-visit record 604; visit record 602-visit record 606; visit record 602-visit record 608; and visit record 610-visit record 606.

Referring to FIG. 6D, a graph data structure 656 representing referred visit paths is shown. The system generates a graph data structure 656 representing referred visit paths. The system generates a vertex list, including a set of vertices representing each of the visit records 650. The vertices are illustrated as nodes for visit record 602, visit record 604, visit record 606, visit record 608, and visit record 608. The system generates an edge list, including a set of edges representing each link between a selected 2-tuple 654. The edges include: visit record 602→visit record 604; visit record 602→visit record 606; visit record 602→visit record 608; and visit record 610→visit record 606.

Referring to FIG. 6E, the graph data structure 656, after addressing problematic vertices, is shown. The system identifies any vertices to which more than one edge connects as problematic vertices. The system determines that visit record 606 is a problematic vertex, because both visit record 602 and visit record 610 connect to visit record 606. Meanwhile, the system determines that visit record 602 is not a problematic vertex. The system permits a vertex from which more than one edge connects.

To address the problematic vertex representing visit record 606, the system determines the visit times of visit record 602 and visit record 610. The system determines that the visit time (February 8) of visit record 610 is more recent than the visit time (February 1) of visit record 602. Therefore, the system keeps the edge between visit record 610 and visit record 606. The system discards the edge between visit record 602 and visit record 606.

The system applies a connected component algorithm to graph data structure 656. The system determines that graph data structure 656 includes two connected components. Each connected component represents a visit path. One visit path includes visit record 602 connected to each of visit record 604 and visit record 608. Another visit path includes visit record 610 connecting to visit record 606.

Referring to FIG. 6F, a graph data structure 658 representing a physician network is shown. The system generates graph data structure 658 based on graph data structure 656. The system determines that all visit paths of graph data structure 656 are of interest. The system traverses each edge in the edge list of graph data structure 656.

The system first identifies an edge, in graph data structure 656, connecting visit record 602 to visit record 604. The system determines that visit record 602 includes physician ID P4, and visit record 604 includes physician ID P5. The system therefore generates, in graph data structure 658, a vertex 612 representing physician ID P4, and a vertex 614 representing physician ID P5. The system generates, in graph data structure 658, an edge connecting physician ID P4 to physician ID P5.

The system then identifies an edge, in graph data structure 656, connecting visit record 602 to visit record 608. The system determines that visit record 602 includes physician ID P4, and visit record 608 includes physician ID P6. The system therefore generates, in graph data structure 658, a vertex 616 representing physician ID P6. The system determines that a vertex representing physician ID P4 already exists in graph data structure 658; hence there is no need to generate another vertex representing physician ID P4. The system generates, in graph data structure 658, an edge connecting physician ID P4 to physician ID P6.

The system then identifies an edge, in graph data structure 656, connecting visit record 610 to visit record 606. The system determines that visit record 610 includes physician ID P4, and visit record 606 includes physician ID P5. The system determines that a vertex representing physician ID P4 and a vertex representing physician ID P5 already exist in graph data structure 658; hence there is no need to generate additional vertices representing physician ID P4 and physician ID P5. The system generates, in graph data structure 658, an edge connecting physician ID P4 to physician ID P5.

The system determines that all edges in graph data structure 656 have been traversed. The system searches through the edge list generated for graph data structure 658 to determine whether any entries refer to the same edge. The system determines that there are two entries referring to an edge connecting physician ID P4 to physician ID P5.

The system generates an aggregated edge list. The system lists, in the aggregated edge list, each unique edge—Physician ID P4→Physician ID P5, and Physician ID P4→Physician ID P6. The system stores, in the aggregated edge list, a count of the occurrences of each edge. The count for Physician ID P4→Physician ID P5 is two. The count for Physician ID P4→Physician ID P6 is one.

In an alternative embodiment, the system directly generates the aggregated edge list, without generating the edge list that may include multiple entries of the same edge. Each time the system determines an edge connecting two physicians to be added to graph data structure 658, the system determines whether the edge already exists in graph data structure 658. If the edge does not already exist in graph data structure 658, then the system adds an entry in the aggregated edge list of graph data structure 658 for the edge. If the edge does already exist in graph data structure 658, then the system does not add any entries in the aggregated edge list, but rather increases a count corresponding to the edge as stored in the aggregated edge list.

Referring to FIG. 6G, a graph data structure 660 representing inferred visit paths is shown. The system identifies a set of criteria for determining a visit path based on implicit referrals. The criteria include: (a) the patient indicated by a visit record in a first element of a 2-tuple is the same as the patient indicated by a visit record in a second element of the 2-tuple; (b) the visit time indicated by the visit record in the first element of the 2-tuple is before the visit time indicated by the visit record in the second element of the 2-tuple; and (c) a difference between the two visit times is below a threshold value. The system applies the criteria to each of the candidate set of 2-tuples 652. The system determines that only four 2-tuples match the criteria: visit record 602-visit record 604; visit record 608-visit record 610; visit record 608-visit record 606; and visit record 610-visit record 606.

The system generates a graph data structure 660 representing inferred visit paths. The system generates a vertex list, including a set of vertices representing each of the visit records 650. The vertices are illustrated as nodes for visit record 602, visit record 604, visit record 606, visit record 608, and visit record 608. The system generates an edge list, including a set of edges representing each link between a selected 2-tuple under the criteria for inferred visit paths. The edges include: visit record 602→visit record 604; visit record 608→visit record 610; visit record 608→visit record 606; and visit record 610→visit record 606.

Referring to FIG. 6H, the graph data structure 660, after addressing problematic vertices, is shown. The system identifies any vertices to which more than one edge connects as problematic vertices. The system determines that visit record 606 is a problematic vertex, because both visit record 608 and visit record 610 connect to visit record 606. Meanwhile, the system determines that visit record 608 is not a problematic vertex. The system permits a vertex from which more than one edge connects.

To address the problematic vertex representing visit record 606, the system determines the visit times of visit record 608 and visit record 610. The system determines that the visit time (February 8) of visit record 610 is more recent than the visit time (February 7) of visit record 608. Therefore, the system keeps the edge between visit record 610 and visit record 606. The system discards the edge between visit record 608 and visit record 606.

The system applies a connected component algorithm to graph data structure 660. The system determines that graph data structure 660 includes two connected components. Each connected component represents a visit path. One visit path includes visit record 602 connected to visit record 604. Another visit path includes visit record 608 connecting to visit record 610, which connects to visit record 606.

Referring to FIG. 6I, a graph data structure 662 representing a physician network is shown. The system generates graph data structure 662 based on graph data structure 660. The system determines that all visit paths of graph data structure 660 are of interest. The system traverses each edge in the edge list of graph data structure 660. For each edge, the system determines a first visit record corresponding to a “source” of the edge and a second visit record corresponding to a “destination” of the edge. The system determines a first physician indicated by the first visit record and a second physician indicated by the second visit record.

The system generates and/or identifies, in graph data structure 662, a first vertex representing the first physician and a second vertex representing the second physician. The system determines that an edge connecting the first vertex to the second vertex is to be added to graph data structure 662. The system keeps track of a count of occurrences of each edge added to graph data structure 662. The system determines that an aggregated edge list includes two unique edges: Physician ID P4→Physician ID P5, and Physician ID P6→Physician ID P4. The system determines that a count for Physician ID P4→Physician ID P5 is two. The system determines that a count for Physician ID P6→Physician ID P4 is one.

FIG. 7A illustrates an example of a graph, presented on a user interface, representing a physician referral network, in accordance with one or more embodiments. A system receives a request to determine which physicians are influential in getting a patient to ultimately see a particular physician of interest 702. The physician of interest 702 may be responsible for a particular medical procedure that a user wishes to study. In response to the request, the system selects referred visit paths and inferred visit paths, of a particular patient, that include a visit record associated with the physician of interest 702. The system may determine that visit paths of interest include portions of referred visit paths ending with a visit record associated with the physician of interest 702. The system may determine that visit paths of interest include portions of inferred visit paths ending with a visit record associated with the physician of interest 702. Based on the visit paths of interest, the system determines a graph data structure representing a physician network. The system causes display of graph 700 representing the physician network.

Graph 700 includes a set of nodes presenting the physicians in the physician network. A particular node represents the physician of interest 702. A size of each node is proportional to a count for edges, in the graph data structure representing the physician network, derived from visit paths of interest that are inferred visit paths. A color of each node represents a provider associated with the physician represented by the node.

Graph 700 includes a set of links connecting the nodes. Each link represents an edge in the graph data structure representing the physician network. A color of each link is proportional to the geographical distance between the physicians represented by the nodes connected by the link. The layout of graph 700 (positions of nodes and lengths of links) is determined by a force-directed graph layout algorithm.

Based on graph 700, a user may determine that a node representing physician 704 is the largest node. The user may determine that physician 704 is involved in the largest number of implicit referrals. Hence, the user may infer that physician 704 is responsible for the general management of the care of the particular patient.

The user may determine that a node representing physician 706 is associated with a different provider from all other nodes. Hence, the user may determine that physician 706 is the main external referral source for directing the particular patient to the provider associated with the physician of interest 702, and ultimately to the physician of interest 702 himself.

The user may identify physicians 704 and 706 as important physicians for leading a patient to ultimately see the physician of interest 702. The user may take actions associated with physicians 704 and 706 that improves the referral pathway to the physician of interest 702. As an example, the user may reach out to physician 706. The user may inform physician 706 of the influential power of physician 704. The user may direct physician 706 to make referrals directly to physician 704. As another example, the user may reach out to physician 704. The user may educate physician 704 about the expertise and experience of the physician of interest 702. The user may encourage physician 704 to make referrals directly to physician 702.

FIG. 7B illustrates an example of a graph, presented on a user interface, representing a relational network on medical departments, in accordance with one or more embodiments.

A system identifies a set of visit paths of interest. Each visit record indicates a medical department associated with the visit. The medical department indicated by a visit record may be the medical department of a physician that sees the patient. Based on the visit paths of interest, the system identifies medical departments indicated by adjacent visits. The system generates a graph data structure representing a relational network on medical departments. The vertices represent the medical departments. The edges represent relationships between the medical departments, as indicated by adjacent visits on a visit path. The system may present graph 708 representing the relational network on medical departments.

As illustrated, graph 708 includes a set of nodes 710 arranged along a ring shape. Each node 710 represents a medical department, such as family practice, internal medicine, hematology & oncology, pulmonary disease, and radiology. Links (such as link 712) between the nodes 710 indicate a relationship between the medical departments represented by the nodes 710. A thickness of a link between two nodes 710 may represent a count of the edges between the medical departments represented by the nodes 710.

### 5. HARDWARE OVERVIEW

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

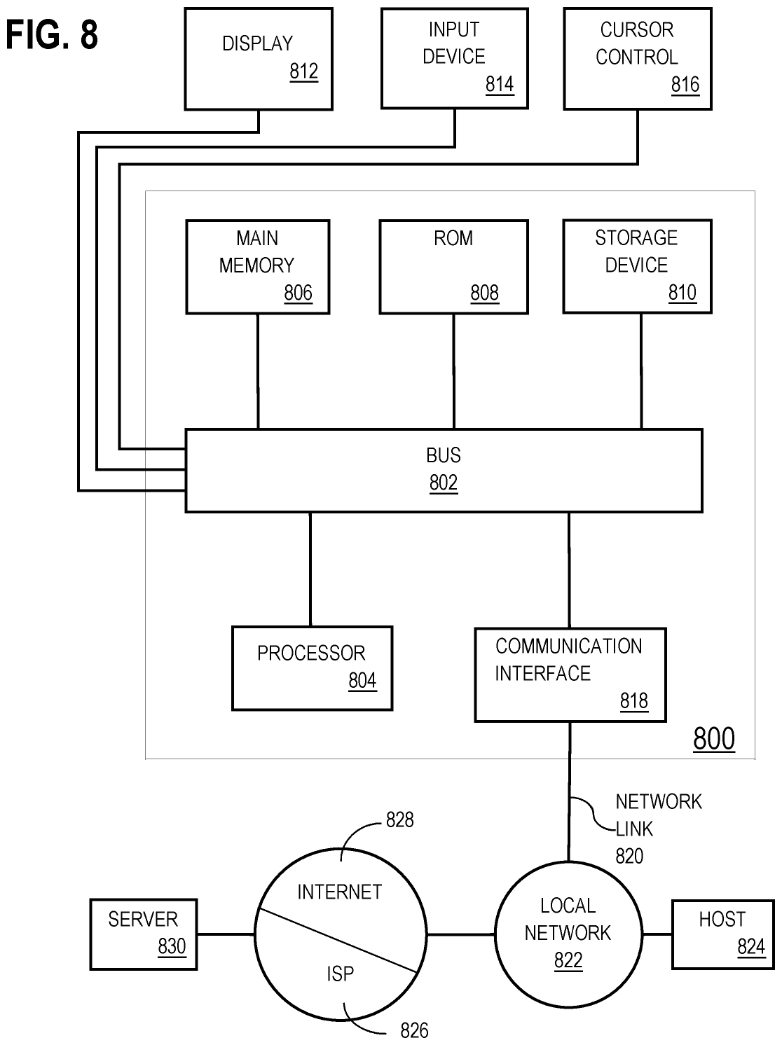

For example, FIG. 8 is a block diagram that illustrates a computer system 800 upon which an embodiment of the invention may be implemented. Computer system 800 includes a bus 802 or other communication mechanism for communicating information, and a hardware processor 804 coupled with bus 802 for processing information. Hardware processor 804 may be, for example, a general purpose microprocessor.

Computer system 800 also includes a main memory 806, such as a random access memory (RAM) or other dynamic storage device, coupled to bus 802 for storing information and instructions to be executed by processor 804. Main memory 806 also may be used for storing temporary variables or other intermediate information during execution of instructions to be executed by processor 804. Such instructions, when stored in non-transitory storage media accessible to processor 804, render computer system 800 into a special-purpose machine that is customized to perform the operations specified in the instructions.

Computer system 800 further includes a read only memory (ROM) 808 or other static storage device coupled to bus 802 for storing static information and instructions for processor 804. A storage device 810, such as a magnetic disk or optical disk, is provided and coupled to bus 802 for storing information and instructions.

Computer system 800 may be coupled via bus 802 to a display 812, such as a cathode ray tube (CRT), for displaying information to a computer user. An input device 814, including alphanumeric and other keys, is coupled to bus 802 for communicating information and command selections to processor 804. Another type of user input device is cursor control 816, such as a mouse, a trackball, or cursor direction keys for communicating direction information and command selections to processor 804 and for controlling cursor movement on display 812. This input device typically has two degrees of freedom in two axes, a first axis (e.g., x) and a second axis (e.g., y), that allows the device to specify positions in a plane.

Computer system 800 may implement the techniques described herein using customized hard-wired logic, one or more ASICs or FPGAs, firmware and/or program logic which in combination with the computer system causes or programs computer system 800 to be a special-purpose machine. According to one embodiment, the techniques herein are performed by computer system 800 in response to processor 804 executing one or more sequences of one or more instructions contained in main memory 806. Such instructions may be read into main memory 806 from another storage medium, such as storage device 810. Execution of the sequences of instructions contained in main memory 806 causes processor 804 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 810. Volatile media includes dynamic memory, such as main memory 806. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 802. Transmission media can also take the form of acoustic or light waves, such as those generated during radio-wave and infra-red data communications.

Various forms of media may be involved in carrying one or more sequences of one or more instructions to processor 804 for execution. For example, the instructions may initially be carried on a magnetic disk or solid state drive of a remote computer. The remote computer can load the instructions into its dynamic memory and send the instructions over a telephone line using a modem. A modem local to computer system 800 can receive the data on the telephone line and use an infra-red transmitter to convert the data to an infra-red signal. An infra-red detector can receive the data carried in the infra-red signal and appropriate circuitry can place the data on bus 802. Bus 802 carries the data to main memory 806, from which processor 804 retrieves and executes the instructions. The instructions received by main memory 806 may optionally be stored on storage device 810 either before or after execution by processor 804.

Computer system 800 also includes a communication interface 818 coupled to bus 802. Communication interface 818 provides a two-way data communication coupling to a network link 820 that is connected to a local network 822. For example, communication interface 818 may be an integrated services digital network (ISDN) card, cable modem, satellite modem, or a modem to provide a data communication connection to a corresponding type of telephone line. As another example, communication interface 818 may be a local area network (LAN) card to provide a data communication connection to a compatible LAN. Wireless links may also be implemented. In any such implementation, communication interface 818 sends and receives electrical, electromagnetic or optical signals that carry digital data streams representing various types of information.

Network link 820 typically provides data communication through one or more networks to other data devices. For example, network link 820 may provide a connection through local network 822 to a host computer 824 or to data equipment operated by an Internet Service Provider (ISP) 826. ISP 826 in turn provides data communication services through the world wide packet data communication network now commonly referred to as the “Internet” 828. Local network 822 and Internet 828 both use electrical, electromagnetic or optical signals that carry digital data streams. The signals through the various networks and the signals on network link 820 and through communication interface 818, which carry the digital data to and from computer system 800, are example forms of transmission media.

Computer system 800 can send messages and receive data, including program code, through the network(s), network link 820 and communication interface 818. In the Internet example, a server 830 might transmit a requested code for an application program through Internet 828, ISP 826, local network 822 and communication interface 818.

The received code may be executed by processor 804 as it is received, and/or stored in storage device 810, or other non-volatile storage for later execution.

### 6. MISCELLANEOUS; EXTENSIONS

Embodiments are directed to a system with one or more devices that include a hardware processor and that are configured to perform any of the operations described herein and/or recited in any of the claims below.

In an embodiment, a non-transitory computer readable storage medium comprises instructions which, when executed by one or more hardware processors, causes performance of any of the operations described herein and/or recited in any of the claims.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. One or more non-transitory machine-readable media storing instructions which, when executed by one or more processors, cause:
generating a first graph data structure representing at least a first visit path of a particular patient at least by:
analyzing a set of data structures representing a plurality of patient visit records to determine a first patient visit record, a second patient visit record, and a third patient visit record reference the same particular patient;
responsive at least to determining that a first attending physician of the first visit record is same as a first referring physician of the second visit record: connecting, in the first graph data structure, a first vertex representing the first patient visit record to a second vertex representing the second patient visit record;
responsive at least to determining that a second attending physician of the second visit record is same as a second referring physician of the third visit record: connecting, in the first graph data structure, the second vertex representing the second patient visit record to a third vertex representing the third patient visit record.

2. The one or more media of claim 1, further storing instructions which, when executed by the one or more processors, cause:
generating a second graph data structure representing a physician referral network at least by:
analyzing the first graph data structure to determine that the first vertex representing the first patient visit record is connected to the second vertex representing the second patient visit record;
responsive at least to determining that the first patient visit record indicates the first attending physician and the second patient visit record indicates the second attending physician: increasing a count associated with a connection, in the second graph data structure, connecting a fourth vertex representing the first attending physician to a fifth vertex representing the second attending physician.

3. The one or more media of claim 2, further storing instructions which, when executed by the one or more processors, cause:
presenting a graph, on a user interface, representing the physician referral network, wherein:
a first node of the graph represents the first attending physician;
a second node of the graph represents the second attending physician;
a link between the first node and the second node represents one or more referrals between the first attending physician and the second attending physician.

4. The one or more media of claim 3, wherein a size of the first node is determined based on a number of connections, in the second graph data structure, associated with the first attending physician.

5. The one or more media of claim 3, wherein a length of the link is determined based on a number of connections, in the second graph data structure, between the first attending physician and the second attending physician.

6. The one or more media of claim 3, wherein a color of the first node is determined based on a healthcare provider associated with the first attending physician.

7. The one or more media of claim 1, further storing instructions which, when executed by the one or more processors, cause:
presenting a graph, on a user interface, representing at least the first visit path, wherein:
a first node of the graph represents the first visit record;
a second node of the graph represents the second visit record;
a link between the first node and the second node represents at least a portion of the first visit path between the first visit record and the second visit record.

8. The one or more media of claim 1, further storing instructions which, when executed by the one or more processors, cause:
determining a set of connected components within the first graph data structure, wherein each of the set of connected components represents a respective visit path of the particular patient.

9. The one or more media of claim 1, further storing instructions which, when executed by the one or more processors, cause:
generating a second graph data structure representing at least a second visit path of the particular patient at least by:
analyzing the set of data structures representing the plurality of patient visit records to determine a fourth patient visit record, a fifth patient visit record, and a sixth patient visit record reference the same particular patient;
responsive at least to determining that a first difference between a first visit time indicated by the fourth visit record and a second visit time indicated by the fifth visit record is below a threshold value: connecting, in the second graph data structure, a fourth vertex representing the fourth patient visit record to a fifth vertex representing the fifth patient visit record;
responsive at least to determining that a second difference between the second visit time indicated by the fifth visit record and a third visit time indicated by the sixth visit record is below the threshold value: connecting, in the second graph data structure, the fifth vertex representing the fifth patient visit record to a sixth vertex representing the sixth patient visit record.

10. The one or more media of claim 9, further storing instructions which, when executed by the one or more processors, cause:
aggregating the first graph data structure and the second graph data structure to generate a third graph data structure representing one or more joint visit paths.

11. The one or more media of claim 10, further storing instructions which, when executed by the one or more processors, cause:
generating a fourth graph data structure representing a physician referral network based on the third graph data structure representing the one or more joint visit paths.

12. The one or more media of claim 1, further storing instructions which, when executed by the one or more processors, cause:
applying entity resolution to a set of data structures representing a plurality of insurance claims to generate the set of data structures representing the plurality of patient visit records;
aggregating a first group of insurance claims associated with a first patient visit to generate the first patient visit record;
aggregating a second group of insurance claims associated with a second patient visit to generate the second patient visit record;
aggregating a third group of insurance claims associated with a third patient visit to generate the third patient visit record.

13. One or more non-transitory machine-readable media storing instructions which, when executed by one or more processors, cause:
obtaining a set of visit records;
identifying a first plurality of 2-tuples of visit records at least by:
determining that a first patient indicated by a first visit record is same as the first patient indicated by a second visit record;
determining that a first visit time indicated by the first visit record is before or simultaneous with a second visit time indicated by the second visit record;
determining that a first attending physician indicated by the first visit record is same as a first referring physician indicated by the second visit record;
identifying a first 2-tuple, of the first plurality of 2-tuples, as having the first visit record in a first element of the first 2-tuple and the second visit record in a second element of the first 2-tuple;
generating one or more graph data structures representing one or more visit paths at least by:
generating a first vertex representing the first visit record in the first element of the first 2-tuple;
generating a second vertex representing the second visit record in the second element of the first 2-tuple;
generating an edge representing at least a portion of a first visit path, of the one or more visit paths, wherein the edge connects the first vertex to the second vertex;
wherein the first visit record indicates a first value for a particular metric, and the second visit record indicates a second value for the particular metric;
generating a graph data structure representing a network associated with the particular metric at least by:
responsive to (a) determining that the first visit record and the second visit record are adjacent to each other on the first visit path and (b) determining that the first visit record indicates the first value for the particular metric and the second visit record indicates the second value for the particular metric: increasing a first count of instances where two adjacent visit records on any visit path, of the one or more visit paths, is associated with the first value for the particular metric and the second value for the particular metric;
presenting, on a user interface, a graph representing the network at least by:
presenting, on the user interface, a plurality of nodes representing values for the particular metric indicated by the set of visit records, wherein a first node represents the first value for the particular metric, and a second node represents the second value for the particular metric;
presenting, on the user interface, a first link between the first node and the second node representing the first count of instances where two adjacent visit records on any visit path, of the one or more visit paths, is associated with the first value for the particular metric and the second value for the particular metric.

14. The one or more media of claim 13, further storing instructions which, when executed by the one or more processors, cause:
generating the one or more graph data structures representing the one or more visit paths further by:
determining that the second visit record in the second element of the first 2-tuple is same as the second visit record in a first element of a second 2-tuple of the first plurality of 2-tuples;
generating a third vertex representing a third visit record in the second element of the second 2-tuple;
generating a second edge representing at least a second portion of the first visit path, of the one or more visit paths, wherein the second edge connects the second vertex to the third vertex;
wherein the third visit record indicates a third value for the particular metric;
generating the graph data structure representing the network associated with the particular metric further by:
responsive to (a) determining that the second visit record and the third visit record are adjacent to each other on the first visit path and (b) determining that the second visit record indicates the second value for the particular metric and the third visit record indicates the third value for the particular metric: increasing a second count of instances where two adjacent visit records on any visit path, of the one or more visit paths, is associated with the second value for the particular metric and the third value for the particular metric;
presenting, on the user interface, the graph representing the network further by:
presenting, on the user interface, a third node of the plurality of nodes representing the third value for the particular metric;
presenting, on the user interface, a second link between the second node and the third node representing the second count of instances where two adjacent visit records on any visit path, of the one or more visit paths, is associated with the second value for the particular metric and the third value for the particular metric.

15. The one or more media of claim 13, further storing instructions which, when executed by the one or more processors, cause:
generating the graph data structure representing the network associated with the particular metric further by:
determining that a first attribute of the first visit path satisfies a particular criterion and a second attribute of a second visit path of the one or more visit paths does not satisfy the particular criterion;
selecting the first visit path for generating the network, without selecting the second visit path for generating the network.

16. The one or more media of claim 13, further storing instructions which, when executed by the one or more processors, cause:
identifying a second plurality of 2-tuples of visit records at least by:
determining that the first patient indicated by a third visit record is same as the first patient indicated by a fourth visit record;
determining that a third visit time indicated by the third visit record is before a fourth visit time indicated by the fourth visit record;
determining that a difference between the third visit time and the fourth visit time is below a threshold value;
identifying a second 2-tuple, of the second plurality of 2-tuples, as having the third visit record in a first element of the second 2-tuple and the fourth visit record in a second element of the second 2-tuple;
generating the one or more graph data structures representing the one or more visit paths further by:
generating a third vertex representing the third visit record in the first element of the second 2-tuple;
generating a fourth vertex representing the fourth visit record in the second element of the second 2-tuple;
generating a second edge representing at least a portion of a second visit path, of the one or more visit paths, wherein the second edge connects the third vertex to the fourth vertex;
wherein the third visit record indicates a third value for the particular metric, and the fourth visit record indicates a fourth value for the particular metric;
generating the graph data structure presenting the network associated with the particular metric further by:
responsive to (a) determining that the third visit record and the fourth visit record are adjacent to each other on the second visit path and (b) determining that the third visit record indicates the third value for the particular metric and the fourth visit record indicates the fourth value for the particular metric: increasing a second count of instances where two adjacent visit records on any visit path, of the one or more visit paths, is associated with the third value for the particular metric and the fourth value for the particular metric;
presenting, on the user interface, the graph representing the network at least by:
presenting, on the user interface, a third node of the plurality of nodes representing the third value for the particular metric;
presenting, on the user interface, a fourth node of the plurality of nodes representing the fourth value for the particular metric;
presenting, on the user interface, a second link between the third node and the fourth node representing the second count of instances where two adjacent visit records on any visit path, of the one or more visit paths, is associated with the third value for the particular metric and the fourth value for the particular metric.

17. The one or more media of claim 16, wherein:
the user interface concurrently presents (a) a first plurality of links, including the first link, that are determined based on the first plurality of 2-tuples rather than the second plurality of 2-tuples and (b) a second plurality of links, including the second link, that are determined based on the second plurality of 2-tuples rather the first plurality of 2-tuples; and
the user interface presents the first plurality of links using a particular interface element type and the second plurality of links using a different interface element type.

18. The one or more media of claim 13, wherein the particular metric comprises at least one of:
an attending physician field associated with the set of visit records, and a healthcare provider field associated with the set of visit records.

19. The one or more media of claim 13, wherein obtaining the set of visit records comprises:
obtaining a plurality of insurance claims from a plurality of data sources;
performing entity resolution on the plurality of insurance claims to identify a respective group of one or more insurance claims that is associated with a respective visit;
aggregating information indicated by each respective group of insurance claims into a respective visit record of the set of visit records.

20. The one or more media of claim 13, wherein a length of the first link is determined based on the first count.

21. The one or more media of claim 13, wherein a size of the first node is determined based on the first count.

22. The one or more media of claim 13, wherein a color of the first node is determined based on a healthcare provider associated with the first value of the particular metric represented by the first node.

23. The one or more media of claim 13, further storing instructions which, when executed by the one or more processors, cause:
generating the one or more graph data structures representing the one or more visit paths further by:
applying a distributed connected component algorithm to the one or more visit paths to associate each of the one or more visit paths with a respective identifier.

24. The one or more media of claim 13, further storing instructions which, when executed by the one or more processors, cause:
identifying a second plurality of 2-tuples of visit records at least by:
determining that a second patient indicated by a third visit record is same as the second patient indicated by a fourth visit record, wherein the first patient and the second patient are different;
determining that a third visit time indicated by the third visit record is before or simultaneous with a fourth visit time indicated by the fourth visit record;
determining that a second attending physician indicated by the third visit record is same as a second referring physician indicated by the fourth visit record;
identifying a second 2-tuple, of the second plurality of 2-tuples, as having the third visit record in a first element of the second 2-tuple and the fourth visit record in a second element of the second 2-tuple;
generating a second set of one or more graph data structures representing a second set of one or more visit paths at least by:
generating a third vertex representing the third visit record in the first element of the second 2-tuple;
generating a fourth vertex representing the fourth visit record in the second element of the second 2-tuple;
generating a second edge representing at least a portion of a second visit path, of the second set of one or more visit paths, wherein the second edge connects the third vertex to the fourth vertex;
wherein the third visit record indicates a third value for a particular metric, and the fourth visit record indicates a fourth value for the particular metric;
generating a second graph data structure representing a second network associated with the particular metric at least by:
responsive to (a) determining that the third visit record and the fourth visit record are adjacent to each other on the second visit path and (b) determining that the third visit record indicates the third value for the particular metric and the fourth visit record indicates the fourth value for the particular metric: increasing a second count of instances where two adjacent visit records on any visit path, of the second set of visit paths, is associated with the third value for the particular metric and the fourth value for the particular metric;
presenting, on the user interface, the graph that represents the network and further represents the second network, wherein presenting the graph further comprises:
presenting, on the user interface, a third node of the plurality of nodes representing the third value for the particular metric;
presenting, on the user interface, a fourth node of the plurality of nodes representing the fourth value for the particular metric;
presenting, on the user interface, a second link between the third node and the fourth node representing the second count of instances where two adjacent visit records on any visit path, of the one or more visit paths, is associated with the third value for the particular metric and the fourth value for the particular metric;
wherein the first link and the second link are concurrently presented.

25. The one or more media of claim 13, wherein the user interface does not concurrently present the first link with any link between any of the plurality of nodes that represents any count associated with any visit record associated with a second patient different than the first patient.
